(19) United States
(12) Patent Application Publication
(10) Pub. No.: US 2023/0312615 A1
(43) Pub. Date: Oct. 5, 2023

(54) METAL-ORGANIC FRAMEWORKS FOR THE STORAGE AND DELIVERY OF HYDROGEN SULFIDE, METHODS OF MAKING AND USES OF SAME

(71) Applicant: CORNELL UNIVERSITY, Ithaca, NY (US)

(72) Inventors: Philip J. Milner, Ithaca, NY (US); Justin J. Wilson, Ithaca, NY (US); Ruth M. Mandel, Ithaca, NY (US); Joshua J. Woods, Ithaca, NY (US); Faith E. Chen, Wilmette, IL (US)

(21) Appl. No.: 18/128,821

(22) Filed: Mar. 30, 2023

Related U.S. Application Data
(60) Provisional application No. 63/325,289, filed on Mar. 30, 2022.

(51) Int. Cl. C07F 7/00; A61K 33/04; A61K 47/30; B01J 20/22

(52) U.S. Cl. CPC C07F 7/003; A61K 33/04; A61K 47/30; B01J 20/226

(57) ABSTRACT

Zr-based metal-organic frameworks (Zr-MOFs) independently comprising the following formula and/or structure: Zr₆O₄(OH)₄(polycarboxylate)₆, and methods of making and using same. In various examples, a method produces a Zr-MOF or Zr-MOFs. In various examples, a Zr-MOF is a hydrogen sulfide (H₂S)-loaded Zr-MOF. In various examples, a method produces a (H₂S)-loaded Zr-MOF or (H₂S)-loaded Zr-MOF. In various examples, a Zr-MOF or Zr-MOFs is/are used to deliver H₂S to an aqueous environment, a solvent, or the like. In various examples, a Zr-MOF or Zr-MOFs is/are used to deliver H₂S to an aqueous environment, a solvent, or the like. In various examples, a Zr-MOF or Zr-MOFs is/are used to deliver H₂S to an individual, such as, for example, an individual suffering from or at risk of an ischemia-reperfusion injury, inflammation, a wound, or the like, or any combination thereof.

### CROSS-REFERENCE TO RELATED APPLICATIONS

This application claims the benefit of U.S. Provisional Patent Application No. 63/325,289, filed Mar. 30, 2022; the contents of the above-identified applications are hereby fully incorporated herein by reference in their entirety.

### STATEMENT REGARDING FEDERALLY SPONSORED RESEARCH

This invention was made with government support under grant number GM138165 awarded by the National Institutes of Health. The government has certain rights in the invention.

### BACKGROUND OF THE DISCLOSURE

Hydrogen sulfide (H₂S) is a colorless, flammable gas with a distinctive “rotten-egg” odor that is pervasive in industrial processes such as petroleum refining, natural gas processing, and wastewater treatment. Because H₂S is an irritant to respiratory linings and a chemical asphyxiant, it has a recommended Immediately Dangerous to Life and Health (IDLH) value of 100 ppm, above which prolonged exposure can be fatal. In addition to being toxic to humans at high concentrations, H₂S impurities in gas streams are highly corrosive to piping and other equipment. In spite of its toxic and corrosive nature, H₂S is also an important gasotransmitter in mammals with proposed roles in calcium homeostasis, long-term potentiation, and mediation of oxidative stress. As such, H₂S has been invoked as a potential therapeutic for numerous disorders, including bone disease, brain injury, inflammation, Parkinson's disease, high blood pressure, cancer, and ischemia-reperfusion injury.

The toxicity and flammability of H₂S limit the viability of inhalation as a therapeutic delivery pathway. Indeed, studies regarding the potential biological role and therapeutic value of H₂S rarely employ this gas directly. Instead, researchers have designed small molecule donors that decompose to generate H₂S or related species under physiological conditions. However, the application of these donors is complicated by the concomitant production of reactive byproducts and other sulfur-based species upon H₂S release. An promising alternative strategy is to design biocompatible porous solids capable of storing and controllably releasing gaseous H₂S, avoiding the formation of biologically-active byproducts. In particular, metal-organic frameworks (MOFs), which are porous materials constructed from organic “linkers” and inorganic “nodes,” are potential materials for the controlled delivery of bioactive molecules. The chemical tunability of MOFs achievable through linker modification and surface functionalization is a potential feature that could potentially provide control over the rate and location of H₂S delivery.

To be useful for H₂S delivery in vivo, MOF-based platforms must meet several criteria, including: (1) construction from non-toxic metals and linkers; (2) stability towards H₂S; (3) high H₂S capacities with controlled release under physiological conditions; and (4) stability under physiological conditions (e.g., serum at 37° C.). However, none of the MOFs that have been studied for H₂S adsorption to date fulfill all of these criteria, as many are limited by framework destruction upon irreversible H₂S binding, low capacities, poor biocompatibility, or harsh H₂S desorption conditions. In addition, many previously studied MOFs are constructed from toxic metals (e.g., Ni, Cr) or complex organic linkers that could lead to unwanted side effects in vivo. For example, the only MOF that has been explored for therapeutic H₂S delivery to date, namely, Zn-MOF-74, fails to meet several of these criteria. It is known that H₂S adsorption in Zn-MOF-74 is completely irreversible, which precludes its application for H₂S delivery. This framework was also found to be highly toxic to HeLa cells. The controlled delivery of H₂S remains an unsolved challenge in medicine and biology.

### SUMMARY OF THE DISCLOSURE

The present disclosure provides, inter alia, Zr-based metal-organic frameworks (Zr-MOFs) comprising polycarboxylate groups, methods of making one or more Zr-based (Zr-MOF(s)), hydrogen sulfide-loaded Zr-based metal-organic frameworks (H₂S-loaded Zr-MOFs), methods of making one or more H₂S-loaded Zr-MOF(s), and methods of H₂S delivery.

In various examples, a Zr-based metal-organic framework (Zr-MOF) comprising the following formula: Zr₆O₄(OH)₄(polycarboxylate)₆. In various examples, the Zr-MOF is not Zr₆O₄(OH)₄(fumarate)₆ (Zr-fum) and/or Zr₆O₄(OH)₄(mesaconate)₆ (Zr-mes). In various examples, the polycarboxylate is independently at each occurrence a group chosen from saturated polycarboxylate groups, unsaturated polycarboxylate groups, straight-chain polycarboxylate groups, branched polycarboxylate groups, cyclic polycarboxylate groups, heterocyclic polycarboxylate groups, aromatic polycarboxylate groups, heteroaromatic polycarboxylate groups, and the like, and any combination thereof. In various examples, the Zr-MOF is completely amorphous, at least partially amorphous and/or crystalline, or completely crystalline. In various examples, the Zr-MOF comprises a plurality of pores. In various examples, the pores have a longest linear dimension perpendicular to the long axis of the pores of from about 0.1 Angstrom(s) (Å) to about 200 Å.

In various examples, a method of making one or more Zr-MOF(s) independently comprising the following formula: Zr₆O₄(OH)₄(polycarboxylate)₆ (e.g., where none of the Zr-MOF(s) is/are Zr₆O₄(OH)₄(fumarate)₆ (Zr-fum) and/or Zr₆O₄(OH)₄(mesaconate)₆ (Zr-mes)), comprises: forming a Zr-MOF reaction mixture comprising: one or more zirconium compound(s), one or more polycarboxylic acid(s), one or more polycarboxylate salt(s), or any combination thereof, one or more basic solvent(s), one or more non-basic solvents(s), or any combination thereof, and optionally, one or more acid modulator(s), heating the reaction mixture, where one or more Zr-MOF(s) is/are formed, and optionally, isolating and/or activating the Zr-MOF(s).

In various examples, a H₂S-loaded Zr-MOF comprises a Zr-based metal organic framework (Zr-MOF) comprising the following formula: Zr₆O₄(OH)₄(polycarboxylate)₆ and H₂S (e.g., where the Zr-MOF comprises H₂S). In various examples, the H₂S-loaded Zr-MOF comprises an H₂S loading of from about 0 millimol H₂S per gram of Zr-MOF (mmol H₂S/g Zr-MOF) to about 10 mmol H₂S/g Zr-MOF. In various examples, the H₂S-loaded Zr-MOF is capable of releasing at least a portion of or all of the H₂S comprised in the H₂S-loaded Zr-MOF.

In various examples, a method of making one or more H₂S-loaded Zr-MOF(s) comprises: forming an H₂S-loading reaction mixture comprising: H₂S, and one or more Zr-MOF(s) independently comprising the following formula: Zr6O4(OH)4(polycarboxylate)6, where the one or more Zr-MOF(s) comprising H₂S (the H₂S-loaded Zr-MOF(s)) is/are formed, and optionally, isolating and/or activating the H₂S-loaded Zr-MOF(s). In various examples, a method further comprises (e.g., after forming the H₂S-loaded Zr-MOF(s)), forming one or more desorbed Zr-MOF(s) (e.g., subjecting the H₂S-loaded Zr-MOF(s) to reduced pressure, heat, an aqueous environment, a solvent, or any combination thereof, thereby forming one or more desorbed Zr-MOF(s), and optionally, isolating and/or activating the desorbed Zr-MOF(s) or the like).

In various examples, a method of H₂S delivery comprises: contacting an aqueous environment, a solvent, or the like, or any combination thereof, with one or more H₂S-loaded Zr-MOF(s), wherein at least a portion of or all of the H₂S comprised by the H₂S-loaded Zr-MOF(s) is released into the aqueous environment, the solvent, or the like, or the combination thereof, and wherein one or more desorbed Zr-MOF(s) is/are formed, and optionally, isolating and/or activating the desorbed Zr-MOF(s). In various examples, the H₂S-loaded Zr-MOF(s) deliver from about 0 micromol per liter (μM) H₂S to about 400 μM H₂S to the aqueous environment, the solvent, or the like, or the combination thereof. In various examples, the aqueous environment is comprised within an individual or a portion of an individual. In various examples, the H₂S-loaded Zr-MOF(s) is/are taken up by a cell or a population of cells, and wherein H₂S is released within the cell or the population of cells. In various examples, the individual is an individual suffering from or at risk of an ischemia-reperfusion injury, inflammation, a wound, or any combination thereof, and wherein delivery treats or prevents the ischemia-reperfusion injury, the inflammation, the wound, or the combination thereof, in the individual.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a fuller understanding of the nature and objects of the disclosure, reference should be made to the following detailed description taken in conjunction with the accompanying figures.

FIG. 1 shows density functional theory-calculated ideal structures of known and new sustainably-derived metal-organic frameworks prepared from fumaric, mesaconic, and itaconic acid as part of this work. Gray, white, red, and pale blue spheres correspond to carbon, hydrogen, oxygen, and zirconium, respectively.

FIG. 2 shows comparison of PXRD patterns of Zr-fum, Zr-mes, and Zr-ita prepared in DMF and in water. The patterns for MOFs prepared in DMF (red) and H₂O (blue) were obtained after activation at 100 degrees Celsius (° C.) under vacuum (<100 mbar) using a Schlenk line and were found to still contain ethanol by ¹H-NMR digestion. Further desolvation at 100° C. under stronger vacuum (<10 mbar) led to partial amorphization of Zr-mes and Zr-ita, but not Zr-fum (purple). The predicted patterns based on the previously reported single crystal X-ray diffraction pattern of Zr-fum and from the DFT calculated ideal structures of Zr-mes and Zr-ita are included for reference (green).

FIG. 3 shows H₂S adsorption (closed circles) and desorption (open circles) isotherms at 25° C. (blue), 40° C. (green), and 55° C. (red) of activated Zr-fum-H₂O, Zr-mes-H₂O, and Zr-ita-H₂O. Solid lines represent fits of the adsorption data to a Langmuir-Freundlich model. A data point was considered equilibrated after <0.01% pressure change occurred over a 45 s (s=second(s)) interval.

FIG. 4 shows differential enthalpies of adsorption (−ΔH_{ads}) for H₂S as a function of uptake for activated Zr-fum-H₂O (blue), Zr-mes-H₂O (red), and Zr-ita-H₂O (green), as determined using the Clausius-Clapeyron equation (eqn (1)) and Langmuir-Freundlich fits in FIG. 3. A data point was considered equilibrated after <0.01% pressure change occurred over a 45 s interval.

FIG. 5 shows 77 K N₂ adsorption isotherms of activated Zr-fum-H₂O, Zr-mes-H₂O, and Zr-ita-H₂O before (blue) and after treatment with H₂S at 25° C. (red). After H₂S adsorption, the MOFs were regenerated at 100° C. under vacuum (<10 mbar) for 48 h (h=hour(s)).

FIG. 6 shows cycling capacities for H₂S adsorption (1 bar, 30° C.) in Zr-fum-H₂O, Zr-mes-H₂O, and Zr-ita-H₂O. The samples were regenerated at 100° C. under high vacuum (<10 mbar) between cycles.

FIG. 7 DFT-calculated structures of H₂S binding in Zr-fum, Zr-mes, and Zr-ita with calculated energies of adsorption (−ΔH_{ads}) indicated. The DFT-calculated structures and energies of H₂O adsorption in all three MOFs are included for comparison. Gray, white, red, yellow, and pale blue spheres correspond to carbon, hydrogen, oxygen, sulfur, and zirconium, respectively.

FIG. 8 shows viability of HeLa cells upon exposure to suspensions of activated Zr-fum-H₂O at a range of concentrations in DMEM supplemented with 10% FBS for 72 h (h =hour(s)) at 37° C. Viabilities were determined by incubating the cells with MTT followed by colorimetric analysis using a microplate reader. Results are reported as the average cell viability of 6 wells/concentration compared to untreated cells from three independent trials, with the standard deviation (SD) reported as the error (±SD).

FIG. 9 shows fluorescence assay (excitation at 340 nm) using the turn-on fluorescence probe dansyl azide (DNS-az) to confirm the release of H₂S from Zr-fum-H₂O (second panel) and Zr-ita-H₂O (third panel) upon submersion in phosphate buffered saline (PBS). The black line corresponds to a control experiment lacking MOF. The data were smoothed by adjacent-averaging (15 points).

FIGS. 10(a)-(d) shows representative fluorescence microscopy images of H₂S release in WSPS-loaded HeLa cells treated with (a) water, (b) 0.05 mg mL⁻¹ H₂S-Zr-fum-H₂O, or (c) 200 mM Na₂S for 1 h at 37° C. (d) Quantification of total cellular fluorescence of HeLa cells incubated under the conditions described. The scale bar indicates 100 μm. ns indicates not significant, ** indicates p<0.01.

FIG. 11 shows hypoxia-reoxygenation model of ischemia-reperfusion injury using H9c2 rat cardiomyoblast cells to confirm the ability of Zr-fum-H₂O to delivery H₂S under physiological conditions. Norm=normoxic conditions; Norm+M=normoxic conditions with activated MOF; R=hypoxia followed by normoxic conditions, simulating reperfusion injury; R +MOF=hypoxia followed by normoxic conditions with activated MOF; R+S−M=hypoxia followed by normoxic conditions with H₂S-loaded Zr-fum-H₂O; R+Na₂S=hypoxia followed by normoxic conditions with Na2S.9H₂O . * indicates p<0.05, *** indicates p<0.001, and ns indicates not significant.

FIG. 12 shows viability of HeLa cells upon exposure to suspensions of activated Zr-mes-H₂O at a range of concentrations in DMEM supplemented with 10% FBS for 72 h at 37° C. Viability was determined by the methods described herein.

FIG. 13 shows viability of HeLa cells upon exposure to suspensions of activated Zr-ita-H₂O at a range of concentrations in DMEM supplemented with 10% FBS for 72 h at 37° C. Viability was determined by the methods described herein.

FIG. 14 (a),(b) shows DRIFTS spectra of free H₂S gas (pink), activated Zr-fum-H₂O (black), and activated Zr-fum-H₂O dosed with 500 mbar of H₂S (blue), 750 mbar of H₂S (purple), and 1000 mbar of H₂S (red). Highlighted in gray boxes are the characteristic stretches of free H₂S gas between 2500 and 2000 cm⁻¹. As expected, these stretches increase in intensity with increasing pressure of H₂S. Highlighted in orange is a broad spectral feature at 2500-2600 cm⁻¹ attributed to H₂S bound inside the MOF via hydrogen-bonding interactions. (a) Full spectra and (b) 2000-3000 cm⁻¹ range.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g., 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%, 0.5% to 2.4%, 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about”, it will be understood that the particular value forms a further disclosure. For example, if the value “about 10” is disclosed, then “10” is also disclosed.

As used herein, unless otherwise indicated, the term “alkyl group” refers to branched or unbranched hydrocarbon groups that include only single bonds between carbon atoms. In various examples, an alkyl group is a C₁ to C₅₀ alkyl group, including all integer numbers of carbons and ranges of numbers of carbons therebetween (e.g., a C₁, C₂, C₃, C₄, C₅, or C₆,). In various examples, an alkyl group is a saturated group. In various examples, an alkyl group is a cyclic alkyl group, e.g., a monocyclic alkyl group or a polycyclic alkyl group. Examples of alkyl groups include, but are not limited to, methyl groups, ethyl groups, propyl groups, isopropyl groups, butyl groups, isobutyl groups, tert-butyl groups, benzyl groups, cyclohexyl groups, adamantyl groups, and the like. In various examples, an alkyl group is unsubstituted or substituted with one or more substituent(s). Examples of substituents include, but are not limited to, various substituents such as, for example, halide groups (—F, —Cl, —Br, and —I), hydroxyl groups, amine groups, nitro groups, cyano groups, isocyano groups, alkoxide groups, alcohol groups, ether groups, ketone groups, carboxylate groups, carboxylic acid groups, ester groups, amide groups, thioether groups, and the like, and any combination thereof.

As used herein, unless otherwise stated, the term “alkenyl group” refers to branched or unbranched hydrocarbons comprising at least one carbon-carbon double bond. Examples of alkenyl groups include, but are not limited to, ethenyl groups, propenyl groups, isopropenyl groups, butenyl groups, isobutenyl groups, tert-butenyl groups, and the like. In various examples, an alkenyl group is a C₂ to C₁₀ alkenyl group, including all integer numbers of carbons and ranges of numbers of carbons therebetween (e.g., C₂, C₃, C₄, C₅, or C₆,). In various examples, an alkenyl group is a cyclic alkenyl group, e.g., a monocyclic alkenyl group or a polycyclic alkenyl group. In various examples, an alkenyl group is unsubstituted or substituted with one or more substituent(s). Examples of substituents include, but are not limited to, various substituents such as, for example, halide groups (—F, —Cl, —Br, and —I), hydroxyl groups, amine groups, nitro groups, cyano groups, isocyano groups, alkoxide groups, alcohol groups, ether groups, ketone groups, carboxylate groups, carboxylic acid groups, ester groups, amide groups, thioether groups, and the like, and any combination thereof.

As used herein, unless otherwise stated, the term “cyclic group” refers to branched or unbranched hydrocarbons, which may be saturated or unsaturated (e.g., comprising at least one carbon-carbon double bond or the like.) In various examples, a cyclic group is a monocyclic group or a polycyclic group. In various examples, a cyclic group (which may be referred to as a heterocyclic group) comprises one or more heteroatom(s) in a ring or rings, if present, of the cyclic group, such as, for example, oxygen, nitrogen (e.g., pyridinyl groups and the like), sulfur, and the like, and any combination thereof.

As used herein, unless otherwise indicated, the term “aromatic group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). In various examples, an aromatic group is a polyaromatic group, such as, for example, a polyaromatic group comprising fused aromatic rings, biaromatic (or biraryl) groups, or a combination thereof.

In various examples, an alkenyl group is unsubstituted or substituted with one or more substituent(s). Examples of substituents include, but are not limited to, various substituents such as, for example, halide groups (—F, —Cl, —Br, and —I), hydroxyl groups, amine groups, nitro groups, cyano groups, isocyano groups, alkoxide groups, alcohol groups, ether groups, ketone groups, carboxylate groups, carboxylic acid groups, ester groups, amide groups, thioether groups, and the like, and any combination thereof. Examples of aromatic groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like. In various examples, an aromatic group (which may be referred to as a heteroaromatic group) comprises one or more heteroatom(s) in the ring or rings, if present, of the aromatic group, such as, for example, oxygen, nitrogen (e.g., pyridinyl groups and the like), sulfur, and the like, and any combination thereof. Examples of aromatic groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent radicals and multivalent radicals, such as, for example, divalent radicals, trivalent radicals, and the like). Illustrative examples of groups include:

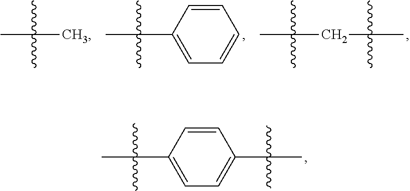

and the like.

The present disclosure provides zirconium (Zr)-based metal-organic frameworks (Zr-MOFs). The present disclosure also provides methods of making and uses of zirconium (Zr)-based metal-organic frameworks (Zr-MOFs).

In an aspect, the present disclosure provides zirconium (Zr)-based metal-organic frameworks (Zr-MOFs). In various examples, a Zr-MOF is made by a method of the present disclosure. Non-limiting examples of Zr-MOFs are disclosed herein.

In various examples, a Zr-based metal-organic framework (Zr-MOF) comprises (or has) the following formula and/or structure: Zr₆O₄(OH)₄(polycarboxylate)₆. In various examples, the Zr₆O₄(OH)₄(polycarboxylate)₆ is Zr₆O₄(OH)₄(itaconate)6 (Zr-ita). In various examples, the Zr₆O₄(OH)₄(polycarboxylate)₆ is not Zr₆O₄(OH)₄(fumarate)6 (Zr-fum) or Zr₆O₄(OH)₄(mesaconate)₆ (Zr-mes).

A Zr-MOF can comprise various polycarboxylates. In various examples, a polycarboxylate (polycarboxylate group) is formed from a polycarboxylic compound or polycarboxylate compound. In various examples, a polycarboxylate (a polycarboxylate group) comprises two or more carboxylate group(s). In various examples, a polycarboxylate is independently at each occurrence chosen from dicarboxylates, tricarboxylates, tetracarboxylates, and the like. In various examples, a polycarboxylate is independently at each occurrence chosen from C₂-C₅₀ polycarboxylates, including all integer carbon number values and ranges therebetween (e.g., a C₁, C₂, C₃, C₄, C₅, or C₆,). In various examples, a polycarboxylate (a polycarboxylate group) comprises one or more alkyl group(s) or the like. In various examples, a polycarboxylate (a polycarboxylate group) comprises one or more alkenyl group(s), which may, independently, comprise terminal (=CH₂) groups, or the like. In various examples, a polycarboxylate (a polycarboxylate group) comprises one or more aromatic group(s) or the like. In various examples, a polycarboxylate (a polycarboxylate group) comprises one or more porphyrin group(s) or the like. In various examples, a polycarboxylate (a polycarboxylate group) comprises one or more alkyl group(s), one or more alkenyl group(s), independently, comprising terminal (=CH₂) groups, one or more aromatic group(s), one or more porphyrin group(s), or the like, or any combination thereof. Non-limiting examples of polycarboxylates include saturated polycarboxylates, unsaturated polycarboxylates, straight-chain polycarboxylates, branched polycarboxylates, cyclic polycarboxylates, heterocyclic polycarboxylates, aromatic polycarboxylates, and heteroaromatic polycarboxylates, and the like, and any combination thereof. In various examples, a polycarboxylate (polycarboxylate group) is independently at each occurrence chosen from carboxylates (carboxylate groups) of fumaric acid, mesaconic acid, itaconic acid, terephthalic acid, succinic acid, glutamic acid, oxalic acid, glutaric acid, 4,4′-biphenyldicarboxylic acid, 2,6-naphthalenedicarboxylic acid, trimesic acid, tetrakis(4-carboxyphenyl)porphyrin, and the like.

A Zr-MOF can comprise a plurality of crystalline and/or amorphous domains. In various examples, a Zr-MOF is completely amorphous, at least partially amorphous and/or crystalline, or completely crystalline.

A Zr-MOF can comprise various porous structures. In various examples, a Zr-MOF comprises a plurality of pores (e.g., a plurality of micropores, a plurality of mesopores, or the like, or any combination thereof), where the pores have a longest linear dimension (e.g., a diameter or the like) perpendicular to the long axis of the pores of from about 0.1 Angstrom(s) (Å) to about 200 Å, including all 0.1 Å values and ranges therebetween. In various examples, a plurality of pores comprises a plurality of micropores having a longest linear dimension of from about 0.1 Å to about 20 Å, including all 0.1 Å values and ranges therebetween, and/or a plurality of mesopores having a longest linear dimension of from about 20 Å to about 150 Å, including all 0.1 Å values and ranges therebetween.

A Zr-MOF can comprise or exhibit various properties. In various examples, a Zr-MOF comprises or exhibits one or more or all of the following: a Brunauer-Emmett-Teller (BET) Surface Area (e.g., as disclosed herein, such as, for example, 77 K N₂) of from about 0 square-meter(s)/gram (m²/g) to about 1000 m²/g, including all 0.1 m²/g values and ranges therebetween (e.g., from about 1 m²/g to about 1000 m²/g, from about 5 m²/g to about 1000 m²/g, from about 10 m²/g to about 900 m²/g, from about 25 m²/g to about 800 m²/g, from about 30 m²/g to about 700 m²/g, from about 45 m²/g to about 600 m²/g, from about 60 m²/g to about 500 m²/g, from about 75 m²/g to about 400 m²/g, or from about 100 m²/g to about 300 m²/g); a Langmuir Surface Area (e.g., as disclosed herein, such as, for example, 77 K N₂) of 0 m²/g to about 1400 m²/g, including all 0.1 m²/g values and ranges therebetween (e.g., from about 1 m²/g to about 1400 m²/g, from about 50 m²/g to about 1400 m²/g, from about 100 m₂/g to about 1200 m²/g, from about 150 m²/g to about 1100 m²/g, from about 200 m²/g to about 1000 m²/g, from about 250 m²/g to about 900 m²/g, from about 300 m²/g to about 800 m²/g, from about 350 m²/g to about 700 m²/g, or from about 400 m²/g to about 600 m²/g); an Enthalpy of Adsorption (−ΔH_{ads}) for hydrogen sulfide (H₂S) (e.g., as disclosed herein, such as, for example, as a function of isosteric H₂S loading using the Clausius-Clapeyron equation, dual-site Langmuir models, and/or Langmuir-Freundlich models) of from about 0 kilojoule(s) (kJ)/mol to about 60 kJ/mol, including all 0.1 kJ/mol values and ranges therebetween (e.g., from about 1 kJ/mol to about 60 kJ/mol, 10 kJ/mol to about 60 kJ/mol, from about 20 kJ/mol to about 55 kJ/mol, from about 25 kJ/mol to about 50 kJ/mol, or from about 30 kJ/mol to about 40 kJ/mol); a H₂S uptake capacity (e.g., as disclosed herein, such as, for example, at 25 degrees Celsius (° C.) and 1 bar of H₂S) of from about 0 millimol H₂S/gram of Zr-MOF (mmol H₂S/g Zr-MOF) to about 10 mmol H₂S/g Zr-MOF, including all 0.01 mmol H₂S/g Zr-MOF values and ranges therebetween (e.g., from about 0.1 mmol H₂S/g Zr-MOF to about 8 mmol H₂S/g Zr-MOF, from about 0.25 mmol H₂S/g Zr-MOF to about 6 mmol H₂S/g Zr-MOF, from about 0.5 mmol H₂S/g Zr-MOF to about 5 mmol H₂S/g Zr-MOF, or from about 1.5 mmol H₂S/g Zr-MOF to about 4.5 mmol H₂S/g Zr-MOF); a H₂S release rate (e.g., a time for a H₂S-loaded Zr-MOF to release sufficient H₂S into an aqueous environment, a solvent, or the like, or any combination thereof, to reach H₂S saturation), of from about less than 30 seconds to less than about 24 hours, including all 1 second values and ranges therebetween; or a H₂S cycling capacity decrease (e.g., as disclosed herein, such as, for example, the reduction of H₂S uptake capacity after multiple cycles of H₂S adsorption (e.g., exposure of a Zr-MOF to H₂S (such as, for example, H₂S gas at 30° C. and 1 bar) and H₂S desorption (e.g., exposure of the H₂S-loaded Zr-MOF to heat and/or vacuum (e.g., at 100° C. at less than 10 μbar)) of from about 0% to about 8%, including all 0.1% values and ranges therebetween after 10 cycles (e.g., from about 0.1% to about 8%, from about 1% to about 8%, from about 2% to about 8%, or from about 4% to about 8%).

In an aspect, the present disclosure provides methods of making Zr-MOFs. A method can make Zr-MOFs of the present disclosure. The methods are based on reaction of one or more zirconium precursor(s) with one or more polycarboxylic acid(s) and/or one or more polycarboxylate salts(s). Non-limiting examples of methods of making Zr-MOFs are disclosed herein.

In various examples, a method comprises making one or more Zr-MOF(s) independently comprising (or having) the following formula and/or structure: Zr₆O₄(OH)₄(polycarboxylate)₆ (e.g., a Zr₆O₄(OH)₄(polycarboxylate)₆ of the present disclosure).

In various examples, two or more or all of the Zr-MOFs are structurally and/or compositionally distinct. In various examples, the Zr₆O₄(OH)₄(polycarboxylate)₆ is Zr₆O₄(OH)₄(itaconate)₆ (Zr-ita). In various examples, the Zr₆O4(OH)₄(polycarboxylate)₆ is not Zr₆O₄(OH)₄(fumarate)₆ (Zr-fum) or Zr₆O₄(OH)₄(mesaconate)₆ (Zr-mes). In various examples, the method of making the Zr-MOF(s) comprises: forming a Zr-MOF reaction mixture comprising: one or more zirconium compound(s) (e.g., ZrOC₁₂, ZrC₁₄, ZrO₂, ZrO(NO₃)₂, a hydrate thereof, or the like, or any combination thereof), one or more polycarboxylic acid(s), one or more polycarboxylate salt(s) (e.g., Na salts, K salts, or the like, or any combination thereof), or the like, or any combination thereof, one or more basic solvent(s) (e.g., N,N-dimethylformamide (DMF), or the like), one or more non-basic solvents(s) (e.g., water at pH about 7.0, ethanol, methanol, or the like, or any combination thereof), or the like, or any combination thereof, and optionally, one or more acid modulator(s) or the like. In various examples, the method further comprises heating the reaction mixture (e.g., for a time and/or a temperature), where Zr-MOF(s) (e.g., of the present disclosure) is/are formed.

A method of making a Zr-MOF can use various reagents in addition to the zirconium compounds and the polycarboxylic acids and/or salt(s) thereof. Nonlimiting examples of additional reagents include solvent(s) and acid modulator(s), and the like, and combinations thereof. In various examples, the solvent(s) do not comprise basic solvent(s). Non-limiting examples of acid modulator(s) include carboxylic acids (such as, for example, formic acid, acetic acid, benzoic acid, substituted benzoic acids, trifluoroacetic acid, pivalic acid, and the like), amino acids (such as, for example, L-proline, histidine, alanine, and the like), mineral acids (such as, for example, hydrochloric acid, nitric acid, sulfuric acid, tetrafluoroboric acid, and the like), and the like, and salts thereof, and any combination thereof.

In various examples, a Zr-MOF reaction mixture comprise from about 1 equivalent(s) (eq) to about 10 eq of polycarboxylic acid(s), polycarboxylate salt(s) (e.g., sodium (Na) salts, potassium (K) salts, or the like, or any combination thereof), or the like, or any combination thereof, including all 0.1 eq values and ranges therebetween, based on the total equivalents of Zr. In various examples, a Zr-MOF reaction mixture comprise from about 0 equivalent(s) (eq) to about 200 eq of acid modulator(s), including all 0.1 eq values and ranges therebetween, based on the total equivalents of Zr.

In various examples, the polycarboxylic acid(s), the polycarboxylate salt(s) (e.g., Na salts, K salts, or the like, or any combination thereof), or the like, or any combination thereof, comprise(s) at least one itaconic acid or itaconate salt, and the solvent(s) do not comprise basic solvent(s). In various examples, the Zr₆O₄(OH)₄(polycarboxylate)₆ is a Zr604(OH)4(itaconate)6 (Zr-ita) and the solvent(s) do not comprise basic solvent(s).

In various examples, the Zr-MOF reaction mixture comprise from about 1 equivalent(s) (eq) to about 10 eq of polycarboxylic acid(s), polycarboxylate salt(s) (e.g., Na salts, K salts, or the like, or any combination thereof), or the like, or any combination thereof, including all 0.1 eq values and ranges therebetween, based on the total equivalents of Zr. In various examples, the Zr-MOF reaction mixture comprise from about 0 equivalent(s) (eq) to about 200 eq of acid modulator(s), including all 0.1 eq values and ranges therebetween, based on the total equivalents of Zr.

In various examples, the Zr-MOF reaction mixture is heated with or without stirring. In various examples, the Zr-MOF reaction mixture is heated with or without refluxing the solvent. In various examples, the Zr-MOF reaction mixture is heated at about the boiling temperature of the solvent(s) (e.g., in a high-pressure reaction vessel, under reflux, or the like).

In various examples, a method comprises various reaction conditions. In various examples, the Zr-MOF reaction mixture is heated with or without stirring. In various examples, the Zr-MOF reaction mixture is heated with or without refluxing the solvent. In various examples, a Zr-MOF reaction mixture is heated at a temperature of from about 30° C. to about 150° C., including all 0.1° C. values and ranges therebetween. In various examples, the Zr-MOF reaction mixture is heated at about the boiling temperature of the solvent(s) (e.g., in a high-pressure reaction vessel, under reflux, or the like).

In various examples, a method comprises various optional process(es). In various examples, a method optionally, further comprises isolating and/or activating the Zr-MOF(s) (e.g., as disclosed herein). In various examples, isolating Zr-MOF(s) is chosen from filtering, centrifuging, rinsing with solvent, solvent-exchanging, and desolvating the Zr-MOF(s), and the like, and any combination thereof. In various examples, rinsing with solvent and/or solvent-exchanging is performed with one or more solvent(s) chosen from alcohols (such as, for example ethanol, and the like, and any combination thereof) and the like.

In various examples, the isolating the Zr-MOF(s) is chosen from filtering, centrifuging, rinsing with solvent, solvent-exchanging, and desolvating the Zr-MOF(s), and the like, and any combination thereof. In various examples, the rinsing with solvent and/or the solvent-exchanging is performed with one or more solvent(s) chosen from alcohols (such as, for example ethanol, and the like, and any combination thereof) and the like.

In various examples, activating Zr-MOF(s) comprises heating the Zr-MOF(s) (e.g., for a time and/or a temperature and/or a pressure). In various examples, activating Zr-MOF(s) comprises heating the Zr-MOF(s) under vacuum at a temperature of from about 30° C. to about 100° C., including all 0.1° C. values and ranges therebetween, and/or at a pressure of from about 10 microbar(s) (μbar) to about 1000 millibar(s) (mbar), including all 0.1 μbar values and ranges therebetween. In various examples, activating the Zr-MOF(s) comprises heating the Zr-MOF(s), where the heating occurs under vacuum (<100 mTorr), with flowing N₂, or the like, or any combination thereof. In various examples, activating the Zr-MOF(s) comprises heating the Zr-MOF(s) with flowing N₂ at a temperature of from about room temperature (e.g., from about 20° C. to about 22° C. or the like) to about 100° C.

In an aspect, the present disclosure provides hydrogen sulfide-loaded Zr-MOFs (H₂S-loaded Zr-MOFs). In various examples, a H₂S-loaded Zr-MOF is made by a method of the present disclosure. In various examples, a H₂S-loaded Zr-MOF comprises a Zr-MOF of the present disclosure and/or a Zr-MOF made by a method of the present disclosure. Non-limiting examples of hydrogen-sulfide loaded Zr-MOFs are described herein.

In various examples, a H₂S-loaded Zr-MOF comprises a Zr-MOF comprising (or having) the following formula and/or structure: Zr₆O₄(OH)₄(polycarboxylate)₆ (e.g., of the present disclosure, such as, for example, according to and/or prepared by a method according to the present disclosure, such as, for example, Zr₆O₄(OH)₄(fumarate)₆ (Zr-fum), Zr₆O₄(OH)₄(mesaconate)₆ (Zr-mes), or Zr₆O₄(OH)₄(itaconate)₆ (Zr-ita)), and H₂S (e.g., where the Zr-MOF comprises H₂S (also known as a H₂S-loaded Zr-MOF, e.g., of the present disclosure, such as, for example, as prepared by a method according to the present disclosure)).

A Zr-MOF of a H₂S-loaded Zr-MOF can comprise H₂S in various portions (or locations) of the Zr-MOF, by various mechanisms, in various quantities, or any combination thereof. In various examples, the H₂S-loaded Zr-MOF comprises H₂S bound to (e.g., disposed on, adsorbed on, or the like, such as, for example, hydrogen bonded to or the like) at least a portion of or all of a surface or a portion of or all of the surfaces of the Zr-MOF (e.g., an external surface, a pore surface, or the like, or any combination thereof). In various examples, a H₂S-loaded Zr-MOF comprises an H₂S loading of from about 0 millimol H₂S/gram of Zr-MOF (mmol H₂S/g Zr-MOF) to about 10 mmol H₂S/g Zr-MOF, including all 0.1 mmol H₂S/g Zr-MOF values and ranges therebetween (e.g., from about 0.1 mmol H₂S/g Zr-MOF to about 8 mmol H₂S/g Zr-MOF, from about 0.25 mmol H₂S/g Zr-MOF to about 6 mmol H₂S/g Zr-MOF, from about 0.5 mmol H₂S/g Zr-MOF to about 5 mmol H₂S/g Zr-MOF, or from about 1.5 mmol H₂S/g Zr-MOF to about 4.5 mmol H₂S/g Zr-MOF).

A H₂S-loaded Zr-MOF can comprise or exhibit various properties. In various examples, a H₂S-loaded Zr-MOF is capable of releasing (e.g., desorbing or the like) at least a portion of or all of the H₂S comprised in the H₂S-loaded Zr-MOF. In various examples, a H₂S-loaded Zr-MOF exhibits a H₂S cycling capacity decrease after 10 cycles (e.g., a cycle as disclosed herein, such as, for example, exposure of a Zr-MOF to 30° C. and 1 bar of H₂S (e.g., adsorption) followed by exposure of the H₂S-loaded Zr-MOF to 100° C. under vacuum (<10 μbar)(e.g., desorption)) of from about 0% to about 8%, including all 0.1% values and ranges therebetween (e.g., from about 0.1% to about 8%, from about 1% to about 8%, from about 2% to about 8%, or from about 4% to about 8%). In various examples, a H₂S-loaded Zr-MOF exhibits a H₂S release rate into an aqueous environment (e.g., the time required for a H₂S-loaded Zr-MOF as disclosed herein to release sufficient H₂S to saturate the aqueous environment) of from about less than 30 seconds to less than about 24 hours, including all 1 second values and ranges therebetween.

In an aspect, the present disclosure provides methods of making H₂S-loaded Zr-MOFs. A method can make H₂S-loaded Zr-MOFs of the present disclosure. The methods are based on reaction of Zr-MOFs with H₂S. In various examples, methods use one or more Zr-MOF(s) of the present disclosure and/or one or more Zr-MOF(s) prepared by methods of the present disclosure. Non-limiting examples of methods of making H₂S-loaded Zr-MOFs are disclosed herein.

In various examples, a method of making one or more H₂S-loaded Zr-MOF(s) comprises: forming an H₂S-loading reaction mixture comprising: H₂S, and one or more Zr-MOF(s) independently comprising (or having) the following formula and/or structure: Zr₆O₄(OH)₄(polycarboxylate)₆ (e.g., as disclosed herein). In various examples, two or more or all of the H₂S-loaded Zr-MOFs are structurally and/or compositionally distinct. In various examples, the Zr₆O₄(OH)₄(polycarboxylate)₆ is Zr₆O₄(OH)₄(fumarate)₆ (Zr-fum), Zr₆O₄(OH)₄(mesaconate)₆ (Zr-mes), Zr₆O₄(OH)₄(itaconate)6 (Zr-ita), or any combination thereof (e.g., according to the present disclosure and/or prepared by a method according to the present disclosure).

A method of making H₂S-loaded Zr-MOF(s) can comprise various reagents and ratios thereof. In various examples, H₂S-loaded MOF(s) comprise(s) H₂S bound to (e.g., disposed on, adsorbed on, or the like, such as, for example, hydrogen bonded to or the like) at least a portion of or all of a surface or a portion of or all of the surfaces of the Zr-MOF (e.g., an external surface, a pore surface, or the like, or any combination thereof)). In various examples, the H₂S-loading reaction mixture comprises H₂S in the gas phase, a solution phase (e.g., as a solution in an organic solvent, such as, for example, tetrahydrofuran or the like), or the like, or any combination thereof. In various examples, the H₂S-loading reaction mixture comprise from about 1 equivalent(s) (eq) to about 100 eq of H₂S, including all 0.1 eq values and ranges therebetween, based on the total equivalents of Zr.

A method of making H₂S-loaded Zr-MOF(s) can comprise various reaction conditions. In various examples, a H₂S-loading reaction mixture is formed with or without stirring. In various examples, Zr-MOF(s) can be dosed with H₂S incrementally.

In various examples, a method of making H₂S-loaded Zr-MOF(s) comprises various optional process(es). In various examples, the method further comprises isolating and/or activating the H₂S-loaded Zr-MOF(s) (e.g., as disclosed herein). In various examples, Zr-MOF(s) will be desolvated and/or activated before H₂S dosing.

In various examples, isolating H₂S-loaded Zr-MOF(s) comprises filtering, centrifuging, rinsing with solvent, solvent-exchanging, or desolvating the H₂S-loaded Zr-MOF(s), or the like, or any combination thereof. In various examples, rinsing with solvent and/or the solvent-exchanging is performed with one or more solvent(s) chosen from alcohols (such as, for example ethanol, and the like, and any combination thereof) and the like.

In various examples, activating H₂S-loaded Zr-MOF(s) comprises heating the H₂S-loaded Zr-MOF(s) (e.g., for a time and/or a temperature and/or a pressure). In various examples, activating H₂S-loaded Zr-MOF(s) comprises heating the H₂S-loaded Zr-MOF(s) under vacuum at a temperature of from about 30° C. to about 100° C., including all 0.1 ° C. values and ranges therebetween, and/or at a pressure of from about 10 microbar(s) (μbar) to about 1000 millibar(s) (mbar), including all 0.1 μbar values and ranges therebetween. In various examples, activating H₂S-loaded Zr-MOF(s) comprises heating the H₂S-loaded Zr-MOF(s), where the heating occurs under vacuum (<100 mTorr), with flowing N₂, or the like, or any combination thereof. In various examples, activating H₂S-loaded Zr-MOF(s) comprises heating the H₂S-loaded MOF(s) with flowing N₂ at a temperature of from about room temperature (e.g., from about 20° C. to about 22° C. or the like) to about 100° C. In various examples, Zr-MOF(s) will be dosed with H₂S prior to activation.

In various examples, the present disclosure provides methods of desorbing H₂S from

H₂S-loaded Zr-MOF(s) of the present disclosure to make desorbed Zr-MOF(s). In various examples, desorbed Zr-MOF(s) is/are used to make H₂S-MOF(s) of the present disclosure. In various examples, a method of making desorbed Zr-MOF(s) further comprises, after forming the H₂S-loaded Zr-MOF(s), forming a desorbed Zr-MOF(s), the method comprising: subjecting the H₂S-loaded Zr-MOF(s) to reduced pressure, heat, an aqueous environment, a solvent, or any combination thereof, thereby forming one or more desorbed Zr-MOF(s), and optionally, isolating and/or activating the desorbed Zr-MOF(s) (e.g., as disclosed herein).

In various examples, desorbed Zr-MOF(s) is/are formed by the removal of at least a portion of, substantially all, or all (e.g., at least about 20%, at least about 30%, at least about 40%, at least about 50%, at least about 60%, at least about 70%, at least about 80%, at least about 90%, or about 100%) of the H₂S from H₂S-loaded Zr-MOF(s). In various examples, desorbed Zr-MOF(s) is/are formed by subjecting H₂S-loaded Zr-MOF(s) to a reduced pressure of from about 10 microbar (μbar) to about 1000 millibar (mbar), including all 0.1 μbar values and ranges therebetween, at a temperature of from about 25° C. to about 100° C., including all 0.1 ° C. values and ranges therebetween. In various examples, desorbed Zr-MOF(s) is/are formed by subjecting H₂S-loaded Zr-MOF(s) to a reduced pressure of less than about 10 mbar at 25 ° C., less than about 10 μbar at 100° C., less than about 1000 mbar at 100° C., or the like.

Desorbed Zr-MOF(s) can comprise various properties. In various examples, a cycle of adsorption of H₂S onto Zr-MOF(s) and desorption of H₂S from H₂S-loaded Zr-MOF(s) can be repeated for up to about 10 cycles without a substantial reduction in H₂S uptake capacity of the desorbed Zr-MOF(s). In various examples, the desorbed Zr-MOF(s) exhibit(s) a H₂S uptake capacity decrease after about 10 cycles (e.g., as disclosed herein, such as, for example, exposure at about 30° C. and about 1 bar of H₂S and desorption at 100 ° C. under vacuum (<about 10 μbar)) of from about 4% to about 8%, including all 0.1% values and ranges therebetween (e.g., from about 0.1% to about 8%, from about 1% to about 8%, from about 2% to about 8%, or from about 4% to about 8%). H₂S-loaded Zr-MOF(s) can comprise various additional process(es). In various examples, isolating desorbed Zr-MOF(s) comprises filtering, centrifuging, rinsing with solvent, solvent-exchanging, or desolvating the desorbed MOF(s), or the like, or any combination thereof. In various examples, rinsing with solvent and/or the solvent-exchanging is performed with one or more solvent(s) chosen from alcohols (such as, for example ethanol, and the like, and any combination thereof) and the like.

In various examples, activating desorbed Zr-MOF(s) comprises heating the desorbed Zr-MOF(s) (e.g., for a time and/or a temperature and/or a pressure). In various examples, activating desorbed Zr-MOF(s) comprises heating the desorbed Zr-MOF(s) under vacuum at a temperature of from about 30° C. to about 100° C., including all 0.1° C. values and ranges therebetween, and/or at a pressure of from about 10 microbar(s) (μbar) to about 1000 millibar(s) (mbar), including all 0.1 μbar values and ranges therebetween. In various examples, the activating the desorbed Zr-MOF(s) comprises heating the desorbed Zr-MOF(s), where the heating occurs under vacuum (<100 mTorr), with flowing N₂, or the like, or any combination thereof. In various examples, activating the desorbed Zr-MOF(s) comprises heating the desorbed Zr-MOF(s) with flowing N₂ at a temperature of from about room temperature (e.g., from about 20° C. to about 22° C. or the like) to about 100° C., including all 0.1° C. values and ranges therebetween.

In an aspect, the present disclosure provides methods of delivery of H₂S. In various examples, methods use H₂S-loaded Zr-MOF(s) of the present disclosure and/or H₂S-loaded Zr-MOF(s) prepared by methods of the present disclosure. Non-limiting examples of methods of delivery of H₂S are disclosed herein.

In various examples, a method of delivery of H₂S comprises: contacting an aqueous environment, a solvent (e.g., tetrahydrofuran, ethanol, dimethyl sulfoxide, or the like, or any combination thereof), or the like, or any combination thereof, with one or more H₂S-loaded Zr-MOF(s) (e.g., as disclosed herein, such as, for example, according to the present disclosure and/or prepared by a method according to the present disclosure), where at least a portion of or all of the H₂S comprised by the H₂S-loaded Zr-MOF(s) is released into the aqueous environment, the solvent, or the like, or any combination thereof, and where desorbed Zr-MOF(s) is/are formed (e.g., as disclosed herein, such as for example, according to the present disclosure), and optionally, isolating and/or activating the desorbed Zr-MOF(s) (e.g., as disclosed herein).

A method of delivery of H₂S can deliver various H₂S concentrations. In various examples, H₂S-loaded Zr-MOF(s) (e.g., about 0.1 mg H₂S-loaded Zr-MOF(s)/mL or less) deliver from about 0 micromol per liter (μM) H₂S to about 400 μM H₂S to the aqueous environment, the solvent, or the like, or any combination thereof (e.g., from about 0.1 μM H₂S to about 400 μM H₂S, from about 0.1 μM H₂S to about 300 μM H₂S, from about 0.1 μM H₂S to about 200 μM H₂S, from about 0.1 μM H₂S to about 100 μM H₂S, or from about 0.1 μM H₂S to about 50 μM H₂S, or about 50 μM H₂S, about 100 μM H₂S, about 200 μM H₂S, about 300 μM H₂S, or about 400 μM H₂S). Non-limiting examples of aqueous environments include deionized water (such as, for example, neutral deionized water and the like), buffered saline (such as, for example, phosphate buffered saline (PBS) and the like), cell culture medium, fetal bovine serum (FBS), and the like, and any combination thereof. In various examples, H₂S-loaded Zr-MOF(s) is/are contacted with the aqueous environment, the solvent, or the like, or any combination thereof, at room temperature, physiological conditions (e.g., about 37° C., or the like), 100° C., or the like. In various examples, H₂S-loaded Zr-MOF(s) is/are subjected to the aqueous environment, the solvent, or the like, or any combination thereof, at a temperature of from about 25° C. to about 100° C., including all 0.1° C. values and ranges therebetween.

In various examples, aqueous environment is disposed within an individual or is a portion of an individual (e.g., cells, tissue, blood, plasma, or the like, or any combination thereof). In various examples, contacting of the portion of an individual with the H₂S-loaded Zr-MOF(s) occurs outside of the individual or within the individual. In various examples, a portion of the individual contacted with the H₂S-loaded Zr-MOF(s) outside of the individual is reintroduced into the individual. In various examples, a H₂S-loaded Zr-MOF(s) is/are taken up by a cell or a population of cells, and where H₂S is released within the cell or the population of cells. In various examples, a cell or the population of cells exhibits greater than about 50% viability (e.g., about 50% or greater, about 60% or greater, about 70% or greater, about 80% or greater, about 90% or greater, or about 100%) after the contacting of the cell or the population of cells with about 1 mg/ml or less of H₂S-loaded Zr-MOF(s).

A method can be used to treat or prevent various conditions. In various examples, an individual is an individual suffering from (or at risk of) an ischemia-reperfusion injury (which may also be referred to as reperfusion injury, reoxygenation injury, hypoxia-ischemia injury, hypoxia-reoxygenation injury, or the like), inflammation (e.g., acute or chronic inflammation, local or systemic inflammation, or the like, or any combination thereof), a wound (e.g., an abrasion, a laceration, a puncture, an avulsion, or the like, or any combination thereof), or the like, or any combination thereof. In various examples, delivery of H₂S to the individual treats or prevents the ischemia-reperfusion injury, the inflammation, the wound, or the like, or any combination thereof, in the individual.

In various examples, an individual is an individual suffering from (or at risk of) developing a disease or condition is an individual diagnosed with a disease or condition, an individual exhibiting one or more symptoms or indicators (e.g., markers or the like) for the disease or condition, an individual having a predisposition for (e.g., genetic factors, co-morbidities, or the like, or any combination thereof) developing the disease or condition, or the like, or any combination thereof. In various examples, an individual is an individual suffering from (or at risk of) developing an ischemia-reperfusion injury has been diagnosed with, exhibits one or more symptoms of, has a predisposition for, or the like, or any combination thereof, one or more or all of the following: a stroke, a myocardial infarction, an organ transplantation, or the like.

In various examples, desorbed Zr-MOF(s) is/are isolated (e.g., from an individual, a portion of an individual, a cell, a population of cells, or the like) and/or activated. In various examples, isolated and/or activated desorbed Zr-MOF(s) are used to make one or more H₂S-loaded Zr-MOF(s) (e.g., as disclosed herein, such as, for example, according to the present disclosure or prepared by a method according to the present disclosure). In various examples, a cycle of adsorption of H₂S onto Zr-MOF(s) and desorption of H₂S from H₂S-loaded Zr-MOF(s) can be repeated for up to about 10 without a substantial reduction in H₂S uptake capacity of the desorbed Zr-MOF(s). In various examples, the desorbed Zr-MOF(s) exhibit(s) a H₂S uptake capacity decrease after about 10 cycles (e.g., as disclosed herein, such as, for example, exposure at about 30° C. and about 1 bar of H₂S and desorption at 100° C. under vacuum (<about 10 μbar)) of from about 4% to about 8%, including all 0.1% values and ranges therebetween (e.g., from about 0.1% to about 8%, from about 1% to about 8%, from about 2% to about 8%, or from about 4% to about 8%).

In various examples, isolating desorbed Zr-MOF(s) (e.g., from an individual, a portion of an individual, a cell, a population of cells, or the like) comprises filtering, centrifuging, rinsing with solvent, solvent-exchanging, or desolvating the desorbed Zr-MOF(s) (e.g., from the individual, the portion of an individual, the cell, the population of cells, or the like, or any combination thereof). In various examples, rinsing with solvent and/or the solvent-exchanging is performed with one or more solvent(s) chosen from alcohols (such as, for example ethanol, and the like, and any combination thereof) and the like.

In various examples, activating desorbed Zr-MOF(s) comprises heating the desorbed Zr-MOF(s) (e.g., for a time and/or a temperature and/or a pressure). In various examples, activating desorbed Zr-MOF(s) comprises heating the desorbed Zr-MOF(s) under vacuum at a temperature of from about 30° C. to about 100° C., including all 0.1° C. values and ranges therebetween, and/or at a pressure of from about 10 microbar(s) (μbar) to about 1000 millibar(s) (mbar), including all 0.1 μbar values and ranges therebetween. In various examples, activating desorbed Zr-MOF(s) comprises heating the desorbed Zr-MOF(s), where the heating occurs under vacuum (<100 mTorr), with flowing N₂, or the like, or any combination thereof. In various examples, activating the desorbed Zr-MOF(s) comprises heating the desorbed Zr-MOF(s) with flowing N₂ at a temperature of from about room temperature (e.g., from about 20° C. to about 22° C. or the like) to about 100° C.

A method of delivery of H₂S to an individual may comprise administering one or more H₂S-loaded Zr-MOF(s) of the present disclosure to the individual (which may be an effective amount of the H₂S-loaded Zr-MOF(s)).

H₂S-loaded Zr-MOF(s)) can be administered to an individual in need thereof via any suitable method or route to an individual in need thereof. Suitable administration routes can include, but are not limited to, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the condition to be treated and/or the active ingredient(s).

An individual (e.g., an individual in need of treatment or the like) may be a human or non-human mammal or other animal. Non-limiting examples of non-human mammals include cows, pigs, mice, rats, rabbits, cats, dogs, and other agricultural mammals, pets (such as, for example, dogs, cats, and the like), service animals, and the like.

“Treating” or “treatment” of any condition refers, in various examples, to ameliorating (e.g., arresting, reversing, alleviating, modulating, or the like) the condition, or reducing the manifestation, extent or severity of one or more clinical symptom(s) thereof, or the like. In various other examples, “treating” or “treatment” refers to ameliorating one or more physical parameter(s), which, independently, may or may not be discernible by the individual. In yet other examples, “treating” or “treatment” refers to modulating the condition, either physically, (e.g., stabilization of one or more discernible symptom(s), or the like), physiologically, (e.g., stabilization of one or more physical parameter, or the like), or both. In yet other examples, treating” or “treatment” relates to slowing the progression of the condition. Treating may include administration of an effective amount of the H₂S-loaded Zr-MOF(s).

Typically, H₂S-loaded Zr-MOF(s) is/are administered in an amount effective to treat a condition, or potential condition as described herein. As used herein, the term “effective amount” means that amount of H₂S-loaded Zr-MOF(s) that will elicit the biological or medical response in an individual that is being sought, for instance, by a researcher, clinician, or the like. Effective doses of the H₂S-loaded Zr-MOF(s) required to treat the progress of the medical condition are readily ascertained by one of ordinary skill in the art using preclinical and clinical approaches familiar to the medicinal arts.

A physician or veterinarian having ordinary skill in the art can readily determine and prescribe the effective amount of H₂S-loaded Zr-MOF(s) required. The selected dosage level can depend upon a variety of factors including, but not limited to, the activity of the particular H₂S-loaded Zr-MOF(s) employed, the time of administration, the rate of excretion or metabolism of the particular H₂S-loaded Zr-MOF(s) being employed, the rate and extent of absorption, the duration of the treatment, other drugs, compounds and/or materials used in combination with the particular H₂S-loaded Zr-MOF(s) employed, the age, sex, weight, condition, general health and prior medical history of an individual being treated, and like factors well known in the medical arts. For example, the physician or veterinarian could start doses of the H₂S-loaded Zr-MOF(s) employed at levels lower than that required in order to achieve the desired therapeutic effect and gradually increase the dosage until the desired effect is achieved.

An effective amount may be a therapeutically effective amount. The term “therapeutically effective amount” includes any amount which, as compared to a corresponding subject who has not received such amount, results in improved treatment, healing, prevention, or amelioration of a disease, disease state, condition, disorder, side effect, or the like or a decrease in the rate of advancement of a disease, disease state, condition, disorder, or the like, or the like. he term also includes within its scope amounts effective to enhance normal physiological function.

An effective amount may result in prophylaxis. The term “prophylaxis” includes prevention and refers to a measure or procedure which is to prevent rather than cure or treat a condition. Preventing may refer to a reduction in risk of acquiring or developing a condition causing at least one clinical symptom of the condition not to develop in an individual that may be exposed to a condition causing agent or a subject predisposed to the condition in advance of condition onset.

The following Statements describe various examples of MOFs, compositions, and methods of the present disclosure and are not intended to be in any way limiting:

Statement 1. A Zr-based metal organic framework (Zr-MOF) comprising (or having) the following formula and/or structure: Zr₆O4(OH)₄(polycarboxylate)₆, excluding Zr₆O₄(OH)₄(fumarate)6 (Zr-fum) or Zr₆O₄(OH)₄(mesaconate)6 (Zr-mes).
Statement 2. A Zr-based metal organic framework (Zr-MOF) according to Statement 1, where the polycarboxylate is independently at each occurrence chosen from saturated, unsaturated, straight-chain, branched, cyclic, heterocyclic, aromatic, and heteroaromatic polycarboxylates, and the like, and any combination thereof.
Statement 3. A Zr-based metal organic framework (Zr-MOF) according to Statement 1 or Statement 2, where the polycarboxylate is independently at each occurrence chosen from carboxylates of fumaric acid, mesaconic acid, itaconic acid, terephthalic acid, succinic acid, glutamic acid, oxalic acid, glutaric acid, 4,4′-biphenyldicarboxylic acid, 2,6-naphthalenedicarboxylic acid, trimesic acid, tetrakis(4-carboxyphenyl)porphyrin, and the like.
Statement 4. A Zr-based metal organic framework (Zr-MOF) according to any one of Statements 1-3, where the Zr-MOF is completely amorphous, at least partially amorphous and/or crystalline, or completely crystalline.
Statement 5. A Zr-based MOF according to any one of Statements 1-4, where the Zr-MOF comprises a plurality of pores (e.g., a plurality of micropores, a plurality of mesopores, or the like, or any combination thereof), where the pores have a longest linear dimension (e.g., a diameter or the like) perpendicular to the long axis of the pores of from about 0.1 Angstrom(s) (Å) to about 200 Å, including all 0.1 Å values and ranges therebetween.
Statement 6. A Zr-based metal organic framework (Zr-MOF) according to any one of Statements 1-5, where the Zr-MOF comprises or exhibits one or more or all of the following: a Brunauer-Emmett-Teller (BET) Surface Area (e.g., as disclosed herein, such as, for example, 77 K N₂) of from about 0 square-meter(s)/gram (m²/g) to about 1000 m²/g, including all 0.1 m²/g values and ranges therebetween; a Langmuir Surface Area (e.g., as disclosed herein, such as, for example, 77 K N₂) of 0 m₂/ to about 1400 m²/g, including all 0.1 m²/g values and ranges therebetween; an Enthalpy of Adsorption (-Aliads) for hydrogen sulfide (H₂S) (e.g., as disclosed herein, such as, for example, as a function of isosteric H₂S loading using the Clausius-Clapeyron equation, dual-site Langmuir models, and/or Langmuir-Freundlich models) of from about 0 kilojoule(s) (kJ)/mol to about 60 kJ/mol, including all 0.1 kJ/mol values and ranges therebetween; a H₂S uptake capacity (e.g., as disclosed herein, such as, for example, at 25° C. and 1 bar of H₂S) of from about 0 millimol H₂S/gram of Zr-MOF (mmol H₂S/g Zr-MOF) to about 10 mmol H₂S/g Zr-MOF, including all 0.01 mmol H₂S/g Zr-MOF values and ranges therebetween; a H₂S release rate (e.g., a time for a H₂S-loaded Zr-MOF to release sufficient H₂S into an aqueous environment, a solvent, or the like, or any combination thereof, to reach H₂S saturation), of from about less than 30 seconds to less than about 24 hours, including all 1 second values and ranges therebetween; or a H₂Scycling capacity decrease (e.g., as disclosed herein, such as, for example, the reduction of H₂S uptake capacity after multiple cycles of H₂S adsorption (e.g., exposure of a Zr-MOF to H₂S(such as, for example, H₂S gas at 30° C. and 1 bar) and H₂S desorption (e.g., exposure of the H₂S-loaded Zr-MOF to heat and/or vacuum (e.g., at 100° C. at less than 10 μbar)) of from about 0% to about 8%, including all 0.1% values and ranges therebetween after 10 cycles.
Statement 7. A method of making one or more Zr-based metal organic framework(s) (Zr-MOF(s)) independently comprising (or having) the following formula and/or structure: Zr₆O₄(OH)₄(polycarboxylate)₆ (e.g., a Zr₆O₄(OH)₄(polycarboxylate)₆ of the disclosure, such as, for example, according to any one of Statements 1-6), excluding Zr6O₄(OH)₄(fumarate)₆ (Zr-fum) or Zr₆O₄(OH)₄(mesaconate)₆ (Zr-mes), the method comprising: forming a Zr-MOF reaction mixture comprising: one or more zirconium compound(s) (e.g., ZrOCl₂, ZrCl₄, ZrO₂, ZrO(NO₃)₂, a hydrate thereof, or the like, or any combination thereof), one or more polycarboxylic acid(s), one or more polycarboxylate salt(s) (e.g., Na salts, K salts, or the like, or any combination thereof), or the like, or any combination thereof, one or more basic solvent(s) (e.g., N,N-dimethylformamide (DMF), or the like), one or more non-basic solvents(s) (e.g., water at pH about 7.0, ethanol, methanol, or the like, or any combination thereof), or the like, or any combination thereof, and optionally, one or more acid modulator(s) or the like, heating the reaction mixture (e.g., for a time and/or a temperature and/or a pressure), where one or more Zr-MOF(s) is/are formed, and optionally, isolating and/or activating the Zr-MOF(s) (e.g., as disclosed herein).
Statement 8. A method according to Statement 7, where the acid modulator(s) is/are chosen from: carboxylic acids (such as, for example, formic acid, acetic acid, benzoic acid, substituted benzoic acids, trifluoroacetic acid, pivalic acid, and the like), amino acids (such as, for example, L-proline, histidine, alanine, and the like), mineral acids (such as, for example, hydrochloric acid, nitric acid, sulfuric acid, tetrafluoroboric acid, and the like), and the like, and any combination thereof.
Statement 9. A method according to Statement 7 or Statement 8, where the Zr-MOF reaction mixture is heated at a temperature of from about 30 degrees Celsius (° C.) to about 150° C., including all 0.1° C. values and ranges therebetween.
Statement 10. A hydrogen sulfide-loaded Zr-based metal organic framework (H₂S-loaded Zr-MOF) comprising a Zr-based metal organic framework (Zr-MOF) comprising (or having) the following formula and/or structure: Zr₆O₄(OH)₄(polycarboxylate)₆ (e.g., of the present disclosure, such as, for example, according to any one of Statements 1-6, such as, for example, as prepared by a method according to any one of Statements 7-9, such as, for example, Zr₆O₄(OH)₄(fumarate)₆ (Zr-fum), Zr₆O₄(OH)₄(mesaconate)6 (Zr-mes), or Zr6O4(OH)4(itaconate)6 (Zr-ita)), where the Zr-MOF comprises H₂S (also known as a H₂S-loaded Zr-MOF, e.g., of the present disclosure, such as, for example, as prepared by a method according to Statement 14). Statement 11. A H₂S-loaded Zr-MOF according to Statement 10, where the Zr-MOF comprises an H₂S loading of from about 0 millimol H₂S per gram of Zr-MOF (mmol H₂S/g Zr-MOF) to about 10 mmol H₂S/g Zr-MOF, including all 0.1 mmol H₂S/g Zr-MOF values and ranges therebetween.
Statement 12. A H₂S-loaded Zr-MOF according to Statement 10 or 11, where the Zr-MOF is capable of releasing (e.g., desorbing or the like) at least a portion of or all of the H₂S comprised in the H₂S-loaded Zr-MOF.
Statement 13. A H₂S-loaded Zr-MOF according to any one of Statements 10-12, where the H₂S-loaded Zr-MOF exhibits a H₂S cycling capacity decrease after 10 cycles (e.g., exposure of a Zr-MOF to 30° C. and 1 bar of H₂S(e.g., adsorption) followed by exposure of the H₂S-loaded Zr-MOF to 100° C. under vacuum (<10 μbar)(e.g., desorption)) of from about 0% to about 8%, including all 0.1% values and ranges therebetween.
Statement 14. A method of making one or more hydrogen sulfide-loaded metal organic framework(s) (H₂S-loaded Zr-MOF(s)), the method comprising: forming an H₂S-loading reaction mixture comprising: H₂S, and one or more Zr-MOF(s) independently comprising (or having) the following formula(s) and/or structure(s): Zr₆O₄(OH)₄(polycarboxylate)₆ (e.g., of the present disclosure, such as, for example, according to any one of Statements 1-6, such as, for example, as prepared by a method according to any one of Statements 7-9, such as, for example, Zr₆O₄(OH)₄(fumarate)₆ (Zr-fum), Zr₆O₄(OH)₄(mesaconate)₆ (Zr-mes), or Zr₆O₄(OH)₄(itaconate)₆ (Zr-ita)), where one or more Zr-MOF(s) comprising H₂S is/are formed (also known as H₂S-loaded Zr-MOF(s), e.g., of the present disclosure, such as, for example, according to any one of Statements 10-13), and optionally, isolating and/or activating the H₂S-loaded Zr-MOF(s) (e.g., as disclosed herein).
Statement 15. A method according to Statement 14, further comprising, after the forming the H₂S-loaded Zr-MOF(s), forming one or more desorbed Zr-MOF(s), the method comprising: subjecting the H₂S-loaded Zr-MOF(s) to reduced pressure, heat, an aqueous environment, a solvent, or any combination thereof, thereby forming one or more desorbed Zr-MOF(s), and optionally, isolating and/or activating the desorbed Zr-MOF(s) (e.g., as disclosed herein). In various examples, one or more desorbed Zr-MOF(s) is/are used to make one or more H₂S-loaded Zr-MOF(s) of the present disclosure, such as, for example, according to any one of Statement 10-13 or prepared by a method according to Statement 14.
Statement 16. A method of delivery of H₂S, the method comprising: contacting an aqueous environment, a solvent (e.g., tetrahydrofuran, ethanol, dimethyl sulfoxide, or the like, or any combination thereof), or the like, or any combination thereof, with one or more H₂S-loaded Zr-MOF(s) (e.g., as disclosed herein, such as, for example, according to any one of Statement 10-13 or prepared by a method according to Statement 14), where at least a portion of or all of the H₂S comprised by the H₂S-loaded Zr-MOF(s) is released into the aqueous environment, the solvent, or the like, or any combination thereof, and where one or more desorbed Zr-MOF(s) is/are formed (e.g., as disclosed herein, such as for example, according to Statement 15), and optionally, isolating and/or activating the desorbed Zr-MOF(s) (e.g., as disclosed herein).
Statement 17. A method according to Statement 16, where H₂S-loaded Zr-MOF(s) (e.g., less than 0.1 mg H₂S-loaded Zr-MOF(s)/mL or the like) deliver from about 0 micromol per liter (μM) H₂S to about 400 μM H₂S, including all 0.1 μM H₂S values and ranges therebetween (e.g., from about 0.1 μM H₂S to about 200 μM H₂S or the like), to the aqueous environment, the solvent, or the like, or any combination thereof.
Statement 18. A method according to Statement 16 or Statement 17, where the aqueous environment is chosen from neutral deionized water, phosphate buffered saline (PBS), cell culture medium, fetal bovine serum (FBS), and the like, and any combination thereof.
Statement 19. A method according to Statement 17 or Statement 18, where, the aqueous environment is comprised within an individual or a portion of an individual (e.g., cells, tissue, blood, plasma, or the like, or any combination thereof).
Statement 20. A method according to any one of Statements 16-19, where the H₂S-loaded Zr-MOF(s) is/are taken up by a cell or a population of cells, and where H₂S is released within the cell or the population of cells.
Statement 21. A method according to Statement 20, where the cell or the population of cells exhibits greater than about 50% viability (e.g., about 50% or greater, about 60% or greater, about 70% or greater, about 80% or greater, about 90% or greater, or about 100%) after the contacting 20 of the cell or the population of cells with about 1 mg/ml or less of H₂S-loaded Zr-MOF(s)).
Statement 22. A method according to Statement 19-21, where the individual is an individual suffering from (or at risk of) an ischemia-reperfusion injury (which may also be referred to as reperfusion injury, reoxygenation injury, hypoxia-ischemia injury, hypoxia-reoxygenation injury, or the like), inflammation (e.g., acute or chronic, local or systemic, or the like), a wound (e.g., an abrasion, a laceration, a puncture, or an avulsion), or the like, or any combination thereof, and where delivery treats or prevents the ischemia-reperfusion injury, the inflammation, the wound, or the like, or any combination thereof, in the individual. In various examples, a desorbed Zr-MOF is used to make a H₂S-loaded Zr-MOF of the present disclosure, such as, for example, according to any one of Statement 10-13 or prepared by a method according to Statement 14.

The steps of the methods described in the various embodiments and examples disclosed herein are sufficient carry out the methods of the present disclosure. Thus, in an embodiment, a method consists essentially of a combination of the steps of the methods disclosed herein. In another embodiment, a method consists of such steps.

The following example is presented to illustrate the present disclosure. It is not intended to be limiting in any manner.

### EXAMPLE

This example provides a description of MOFs and methods of the present disclosure.

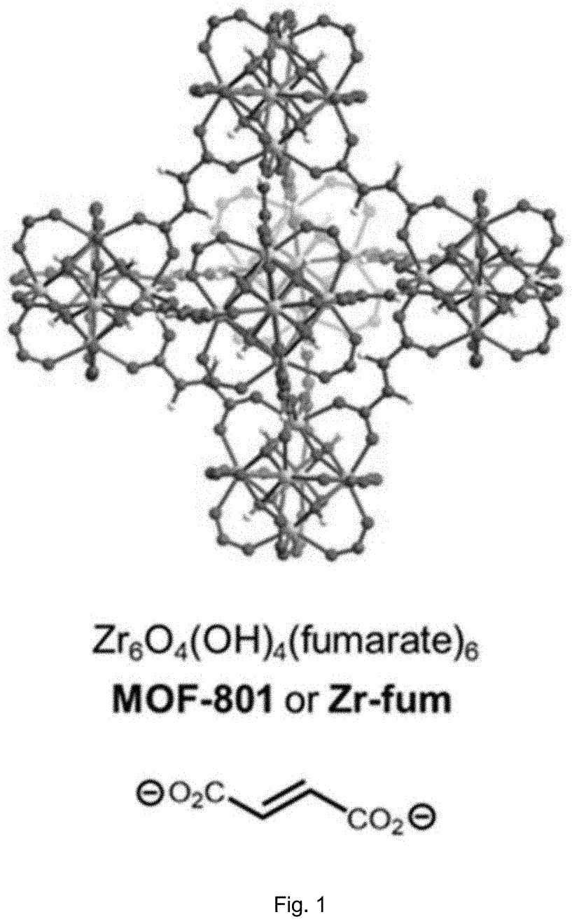

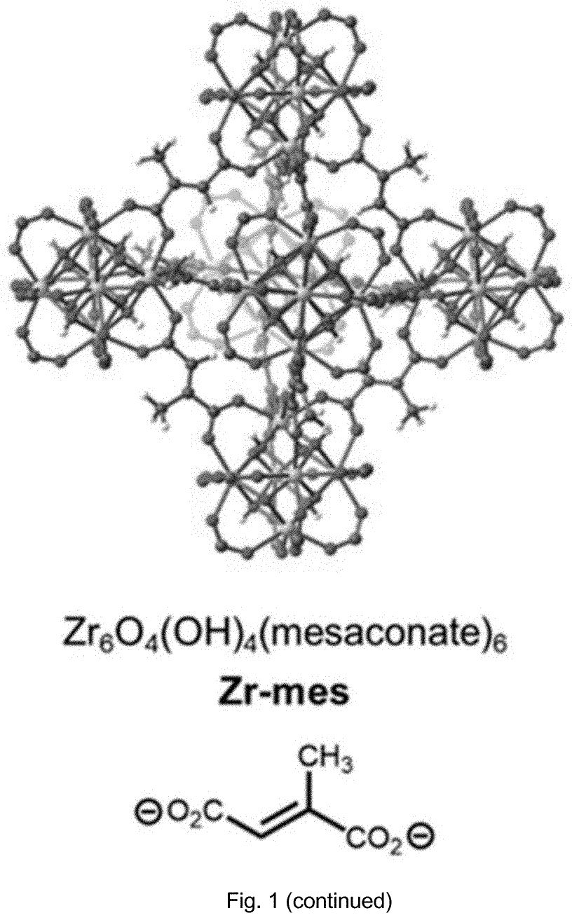

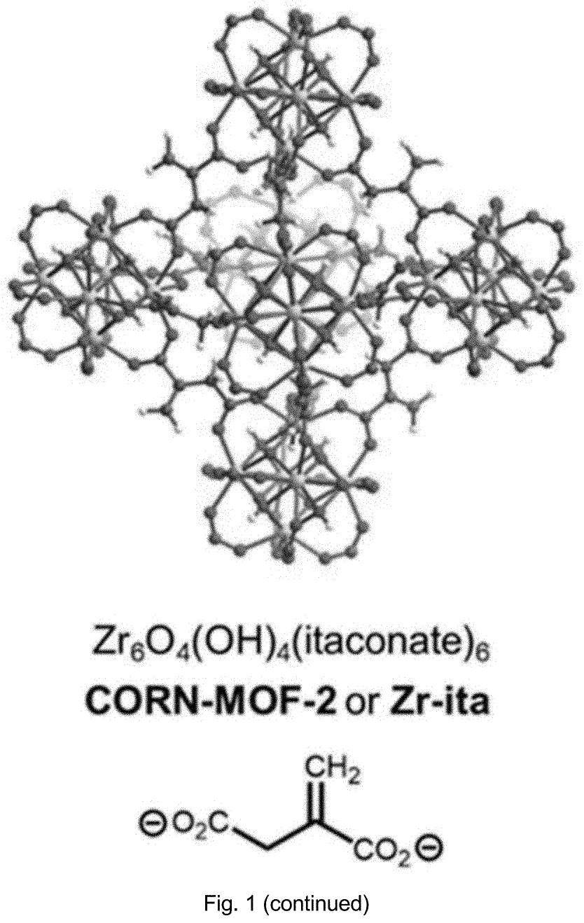

A strategy for controlled delivery of H₂S is to design biocompatible porous solids capable of storing and controllably releasing gaseous H₂S, avoiding the formation of biologically-active byproducts. A strategy to identify new MOFs capable of reversibly adsorbing H₂S is to leverage its properties as a hydrogen-bond donor and weak hydrogen-bond acceptor, similar to water. In particular, we hypothesized that the surprisingly strong binding of water −ΔH_{ads} of approximately 60 kJmol⁻¹) in Zr₆O₄(OH)₄(fumarate)₆ or MOF-801 (FIG. 1, first sheet), which is due to ordered hydrogen-bonding interactions within the tetrahedral cavities, may translate to strong binding of H₂S within this MOF as well. In addition, Zr is a nontoxic metal and fumaric acid occurs naturally as part of the Krebs cycle, suggesting that MOF-801 and closely related frameworks should also be biocompatible.

Through combined experimental and computational analyses we demonstrated that MOF-801, as well as the related biocompatible mesaconate-based framework Zr-mes and itaconate-based framework CORN-MOF-2 (CORN=Cornell University) undergo strong but reversible adsorption of H₂S with minimal framework degradation (FIG. 1). In addition, these frameworks are capable of delivering H₂S under physiologically relevant conditions, allowing for the therapeutic benefits of H₂S to be accessed using easily-handled solids. Taken together, these features make the frameworks described here promising H₂S donors for biomedical applications.

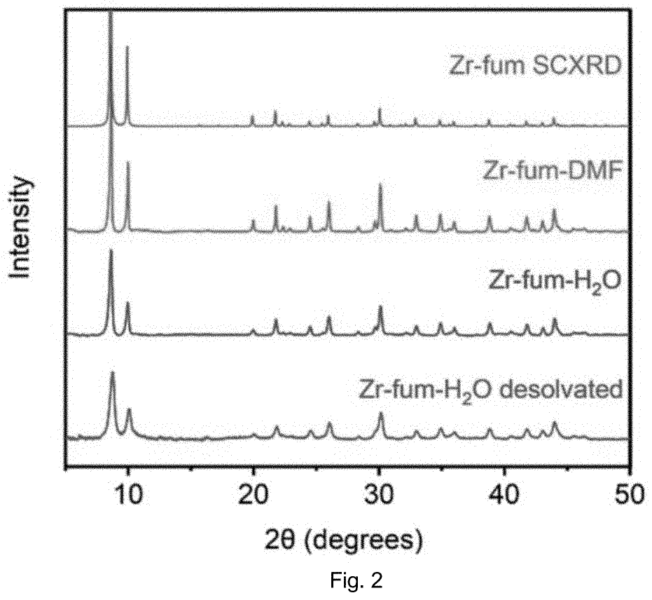

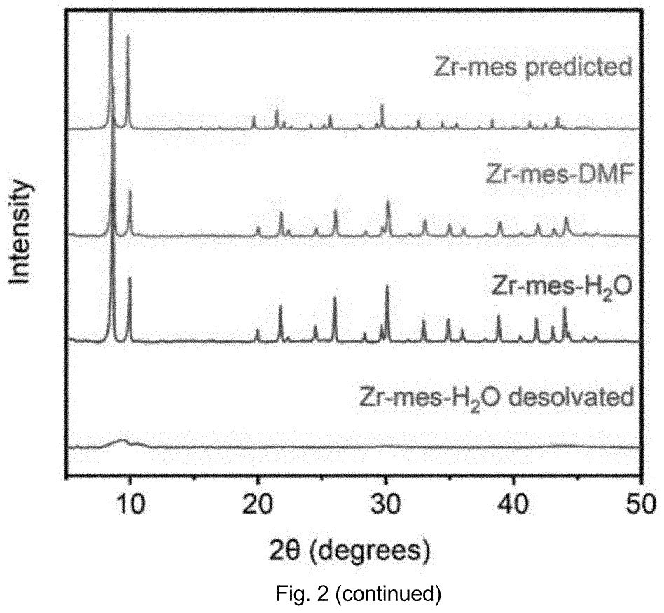

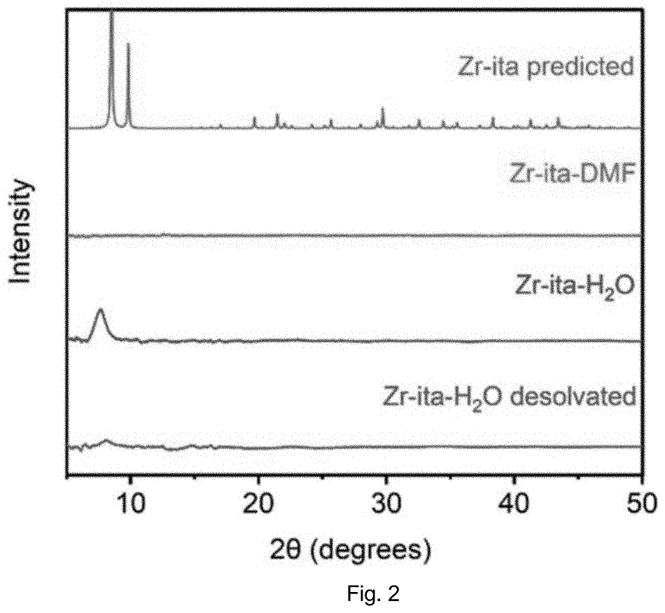

Results and Discussion. Preparation of MOFs. The MOFs Zr₆O₄(OH)₄(fumarate)₆ and Zr₆O₄(OH)₄(mesaconate)₆ are also known as MOF-801 and Zr-mes, respectively. We refer to these frameworks as Zr-fum and Zr-mes, respectively, for clarity. Following procedures adapted from the literature, we prepared Zr-fum and Zr-mes under solvothermal conditions on 10 mmol scale. Specifically, these frameworks were synthesized from ZrOCl₂·8H₂O and the corresponding dicarboxylic acid in either N,N-dimethylformamide (Zr-fum-DMF, Zr-mes-DMF) or water (Zr-fum-H₂O, Zr-mes-H₂O) using formic acid as a modulator. After optimization, 30 equivalents of formic acid were found to produce Zr-fum and Zr-mes with high crystallinity, as determined by powder X-ray diffraction (PXRD). Notably, higher concentrations of formic acid led to the formation of significant impurities. The PXRD patterns of solvated Zr-fum and Zr-mes prepared in DMF and in H₂O are shown in FIG. 2. The PXRD patterns for Zr-fum-DMF and Zr-fum-H₂O match the predicted pattern corresponding to the single-crystal X-ray diffraction (SCXRD) structure of MOF-801. Likewise, the patterns for Zr-mes match the predicted pattern based on the density functional theory (DFT)-calculated ideal structure of this framework. The Langmuir surface areas determined from 77 K N₂ adsorption isotherms of all four frameworks are in good agreement with previously reported values. Unlike Zr-fum-H₂O, Zr-mes-H₂O was found to undergo partial amorphization upon complete desolvation under high vacuum (<10 mbar) at 100° C., although this did not impact its porosity. Notably, synthesis under aqueous conditions produced nanocrystals of Zr-fum-H₂O less than 100 nm in size, suitable for applications in medicine, whereas synthesis in DMF produced larger crystallites approximately 500 nm in size. The framework Zr-mes was obtained as crystallites of intermediate size (100-200 nm) under both sets of conditions. Owing to the similar properties of frameworks prepared in water and in DMF, we employed Zr-fum-H₂O and Zr-mes-H₂O for subsequent studies to bypass the use of toxic DMF. After preparing Zr-fum-H₂O and Zr-mes-H₂O, we next investigated whether the naturally-derived and inexpensive dicarboxylic acid itaconic acid can be used to synthesize an isostructural MOF, which we term CORN-MOF-₂ and refer to as Zr-ita herein for consistency (FIG. 1). Produced via the fermentation of carbohydrates by fungi from the genus Aspergillus, itaconic acid is a desirable linker because of its very low cost and lack of toxicity. Unfortunately, solvothermal synthesis using ZrCl₄ and itaconic acid in DMF produced amorphous material, as confirmed by PXRD (FIG. 2). Several acid modulators were evaluated to enhance the crystallinity of Zr-ita-DMF, including acetic acid, L-proline, concentrated HCl, pivalic acid, and formic acid, but none led to crystalline MOF. In addition, N₂ sorption isotherms of Zr-ita-DMF prepared without an acid modulator revealed minimal porosity and a Brunauer-Emmett-Teller (BET) surface area of only 33 m²g⁻¹. Insight into the poor quality of amorphous Zr-ita-DMF was obtained from 1H-NMR spectra of samples digested using a saturated K₃PO₄/D₂O solution in DMSO-d₆. Peaks due to itaconate and residual solvent were observed as expected. However, additional peaks ascribed to the isomeric mesaconate were also observed. We hypothesized that basic N,N-dimethylamine generated by the hydrolysis of DMF mediates the isomerization of itaconate to mesaconate during framework self-assembly, leading to an amorphous structure. In addition, residual DMF was observed by ¹H-NMR even after extensive soaking in tetrahydrofuran. Therefore, the significant linker isomerization and difficulty in removing DMF from the pores together account for the low surface area of Zr-ita-DMF.

We hypothesized that the key to preparing high-quality Zr-ita was to avoid the use of base during MOF synthesis. Consistently, employing water as the solvent instead of DMF produced semi-crystalline Zr-ita-H₂O for the first time (FIG. 2). The modest crystallinity of Zr-ita-H₂O compared to Zr-fum-H₂O and Zr-mes-H₂O is likely due to the sp3-hybridized carbon in the backbone of this flexible linker. Because of these characteristics, Zr-ita-H₂O is likely an amorphous MOF (aMOF), which have found application for toxic gas capture and drug delivery. Because the DFT-calculated structure of Zr-ita-H₂O is idealized and does not account for linker flexibility, the PXRD pattern simulated from this structure in FIG. 1 does not reflect the amorphous nature of Zr-ita-H₂O. When prepared on 10 mmol scale, Zr-ita-H₂O exhibited improved porosity compared to Zr-ita-DMF, with a 77 K N₂ BET surface area of 235±2 m²g⁻¹ and a Langmuir surface area of 487±22 m²g⁻¹. Marked hysteresis was also observed upon N₂ desorption in Zr-ita-H₂O. Analysis of the pore size distribution from the N₂ sorption isotherm revealed micropores approximately 6.6 Å in diameter, as expected based on the idealized structure, along with mesopores 20-100 Å in size. Thus, we attribute the observed hysteresis to mesoporous defects or cavities between particles. Consistent with the hypothesis outlined above, the ¹H-NMR spectra of Zr-ita-H₂O digested using a saturated K₃PO₄/D₂O solution in DMSO-d₆ revealed only resonances attributed to the itaconate linker, residual formate from defect sites, and solvent. To further illustrate the scalability of Zr-ita-H₂O, we prepared this new framework on 0.5 mol scale, resulting in a total yield of approximately 87 g (73% yield) of activated framework. Thus, Zr-ita-H₂O was employed for subsequent H₂S adsorption studies to compare with Zr-fum-H₂O and Zr-mes-H₂O.

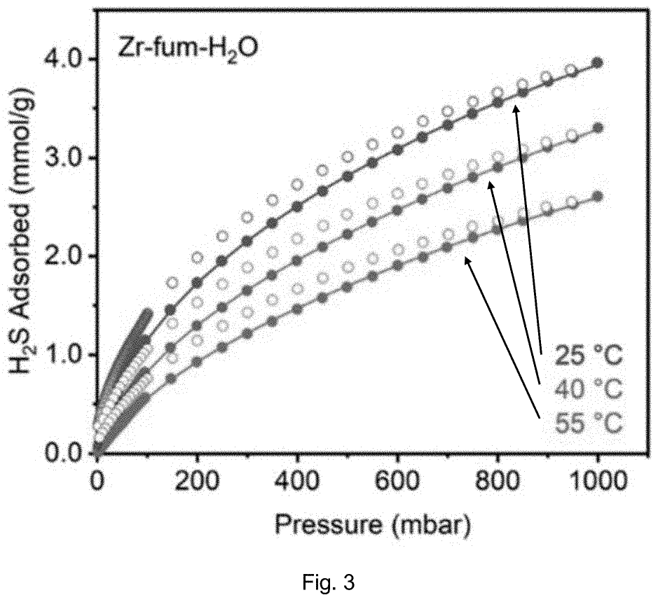

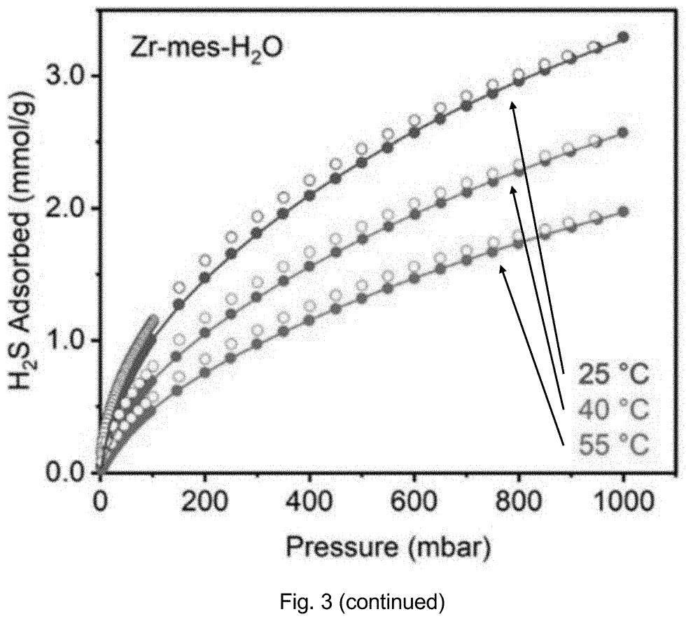

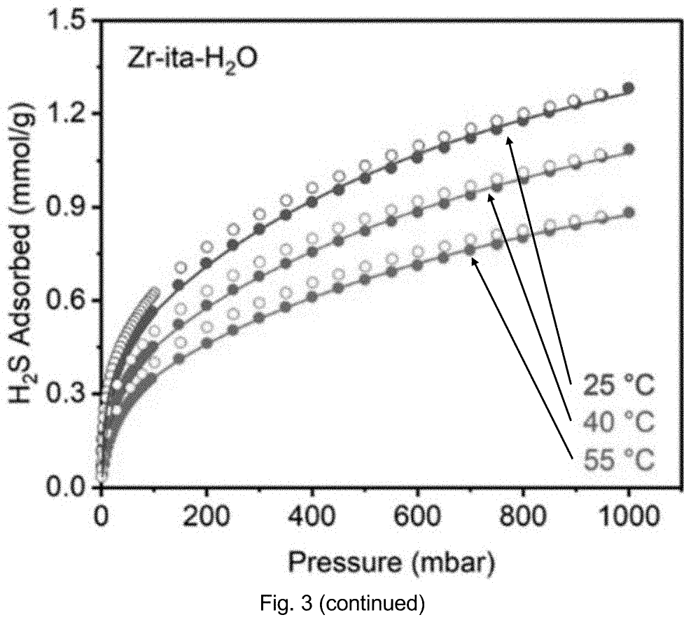

H₂S adsorption in MOFs. After preparing Zr-fum-H₂O, Zr-mes-H₂O, and Zr-ita-₂O, we next evaluated the reversible H₂S capture performance of all three frameworks by carrying out isothermal adsorption measurements at 25° C., 40° C., and 55° C. (FIG. 3). At 25° C. and 1 bar of H₂S, Zr-fum-H₂O displayed the highest H₂S uptake (4.0 mmol g⁻¹1, 11 wt %) followed by Zr-mes-H₂O (3.3 mmol g 1, 10 wt %) and Zr-ita-H₂O (1.3 mmol g^{-1,} 4.0 wt %), which trends with the frameworks' respective surface areas. These measurements indicate that Zr-fum-H₂O and Zr-mes-H₂O possess capacities comparable to those in MOFs that undergo (partially) reversible H₂S adsorption under similar conditions, such as UiO-66 (˜3.0 mmol g⁻; UiO=Universiteteti Oslo), MIL-53 (Al) (3.5 mmol g⁻¹), MIL-53 (Cr) (˜3.5 mmol g⁻¹), and Ga-soc-MOF-1a (˜4.5 mmol g⁻¹; soc=square octahedral). Moreover, the relatively smooth overlay of the adsorption and desorption data suggests that H₂S binds reversibly at every temperature. Although H₂S adsorption is readily reversible at 25° C. in all three frameworks, they were regenerated at 100° C. under high vacuum (<10 mbar) between experiments for consistency. To investigate the thermodynamics of H₂S binding in Zr-fum-H₂O, Zr-mes-H₂O, and Zr-ita-H₂O, we fit the adsorption data in FIG. 3 using both dual-site Langmuir and Langmuir-Freundlich models. Although both models provided adequate fits to the data, the empirical Langmuir-Freundlich fits (FIG. 3) were found to be superior and thus were employed for subsequent analysis. The differential enthalpies of adsorption were calculated as a function of isosteric H₂S loading using the Clausius-Clapeyron equation (Eq. 1), in which PQ is the pressure at a constant uptake Q, −ΔH_{ads} is the differential enthalpy of adsorption, R is the ideal gas constant, T is the temperature, and c is a constant.

$$ln ( {P}_{Q} ) = ( \frac{Δ {H}_{ads}}{R} ) ( \frac{1}{T} ) + c \qquad Eq . 1$$

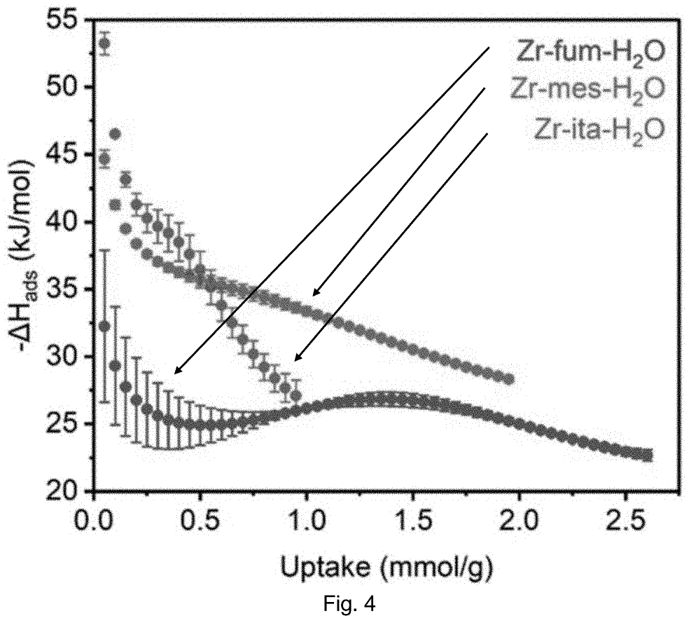

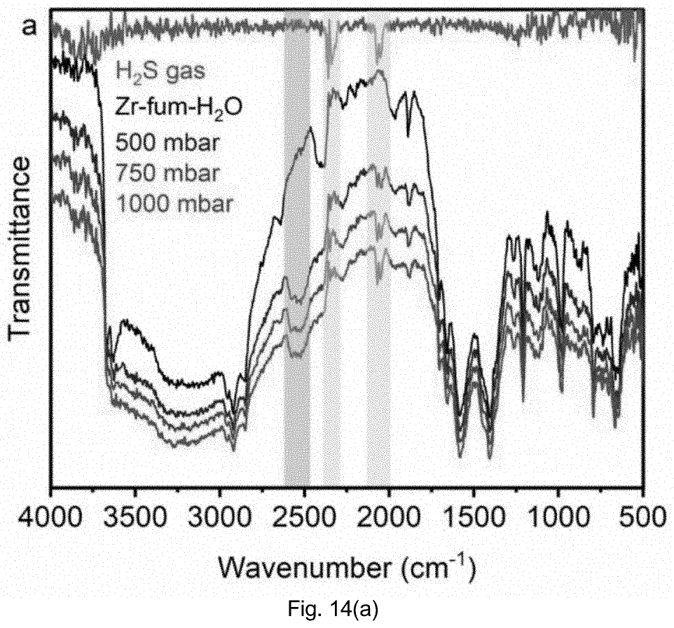

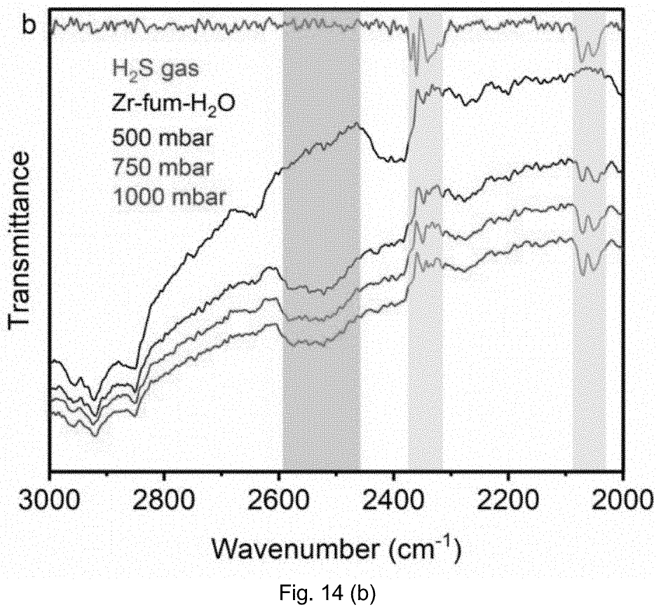

The differential enthalpies of adsorption (−ΔH_{ads}) for all three frameworks are included in FIG. 4. The low-coverage adsorption enthalpies follow the trend Zr-ita-H₂O (53.2±0.8 kJ mol⁻¹) >Zr-mes-H₂O (44.7±0.6 kJ mol⁻¹)>Zr-fum-H₂O (32.2±5.6 kJ mol⁻¹). The same trend was observed when the data were fit using dual-site Langmuir models as well. This difference is likely due to the smaller pores of Zr-ita-H₂O and Zr-mes-H₂O, which enable enhanced hydrogen-bonding interactions between H₂S and the framework. Consistently, preliminary in situ diffuse reflectance infrared Fourier transform spectroscopy (DRIFTS) measurements of H₂S-dosed Zr-fum-H₂O are indicative of hydrogen-bonding interactions between H₂S and the framework upon adsorption (FIG. 14(a),(b)). The data in FIG. 4 are among the first experimentally derived differential enthalpies of H₂S adsorption in MOFs, and they are comparable to or higher than those predicted computationally for previously-reported frameworks. The comparatively strong binding of H₂S in Zr-fum-H₂O, Zr-mes-H₂O, and Zr-ita-H₂O bodes well for their ability to store H₂S prior to release upon exchange with water under physiological conditions.

The reversibility of H₂S adsorption and long-term stability towards H₂S are also critical criteria for prospective H₂S-storage materials. As such, we carefully evaluated the stability of Zr-fum-H₂O, Zr-mes-H₂, and Zr-ita-H₂O towards H₂S in both the gas and solution phases. To characterize the stability of Zr-fum-H₂O, Zr-mes-H₂O, and Zr-ita-H₂O to H₂S in solution, we soaked each MOF in a 0.8 M solution of H₂S in THF at 50° C. for 48 h. Negligible changes in the PXRD patterns, infrared spectra, and 1H-NMR spectra after digestion with a saturated K₃PO₄/D₂O solution were observed for the MOFs after exposure to H₂S in solution, suggesting that, in contrast to Zn-MOF-74, little-to-no framework degradation occurred under these conditions.

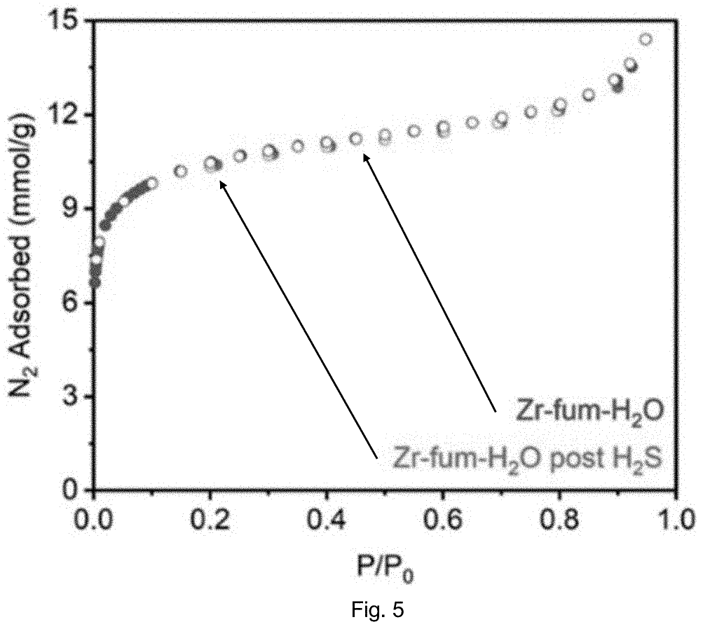

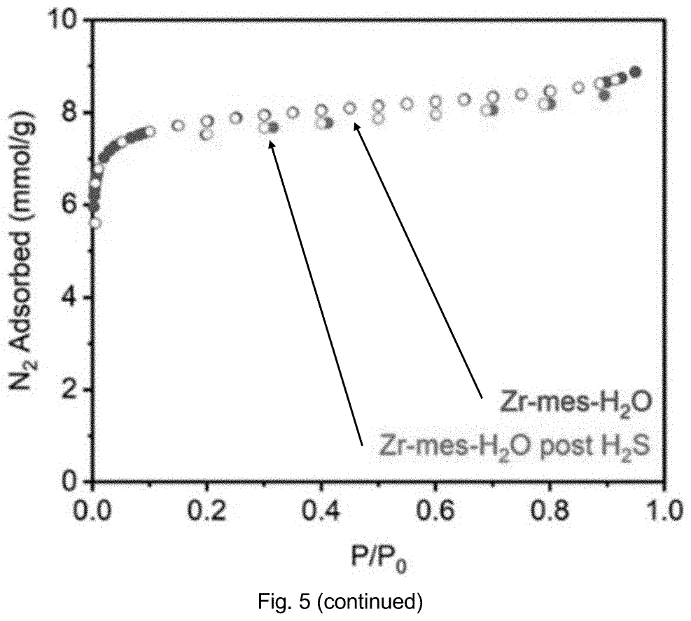

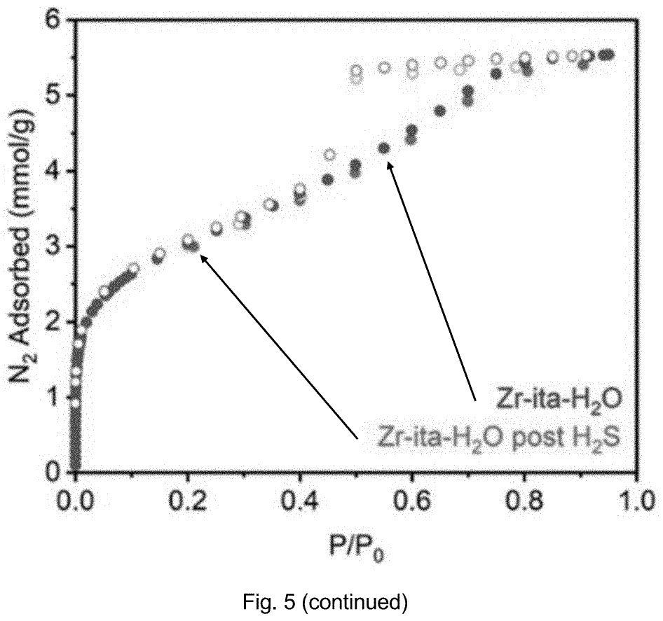

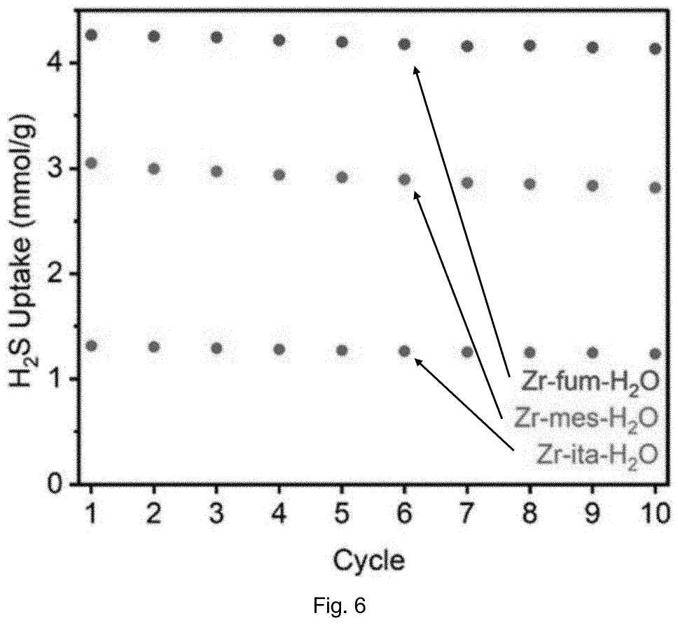

Consistent with the desorption data in FIG. 3, the 77 K N₂ Langmuir surface areas of all three MOFs also remained essentially unchanged after exposure to gaseous H₂S at 25° C. (FIG. 5). In contrast, water vapor binds strongly and only semireversibly to all three frameworks, leading to a partial reduction in their Langmuir surface areas after exposure to H₂O vapor at 35° C. To corroborate the reversibility of H₂S adsorption in Zr-fum-H₂O, Zr-mes-H₂O, and Zr-ita-H₂O, we also carried out cycling experiments in which the MOFs were repeatedly exposed to approximately 1 bar of H₂S at 30° C. followed by desorption at 100° C. under vacuum (FIG. 6). The H₂S cycling capacities of all three frameworks remained relatively constant over ten cycles, decreasing by only 3% for Zr-fum-H₂O, 8% for Zr-mes-H₂O, and 6% for Zr-ita-H₂O, respectively. In addition, 77 K N₂ adsorption measurements confirmed that the Langmuir surface areas of all three frameworks remained high after repeated cycling of H₂S, and characterization by PXRD and ¹H-NMR after digestion with a saturated K₃PO₄/D₂O solution confirmed that minimal degradation occurred during H₂S cycling. Last, the lack of residual sulfur species in Zr-fum-H₂O that had been exposed to H₂S and subsequently activated under vacuum was confirmed by X-ray photoelectron spectroscopy. Together, these experimental measurements confirm H₂S adsorbs reversibly and nondestructively within Zr-fum-H₂O, Zr-mes-H₂O, and Zr-ita-H₂O, in contrast to Zn-MOF-74. Indeed, the excellent performance of these materials upon H₂S cycling places them among the upper echelon of frameworks that have been studied for H₂S capture and/or delivery to date. Importantly, the lack of degradation upon H₂S adsorption in these materials indicates that they should be capable of cleanly delivering pure H₂S that is uncontaminated by any other sulfur-containing species.

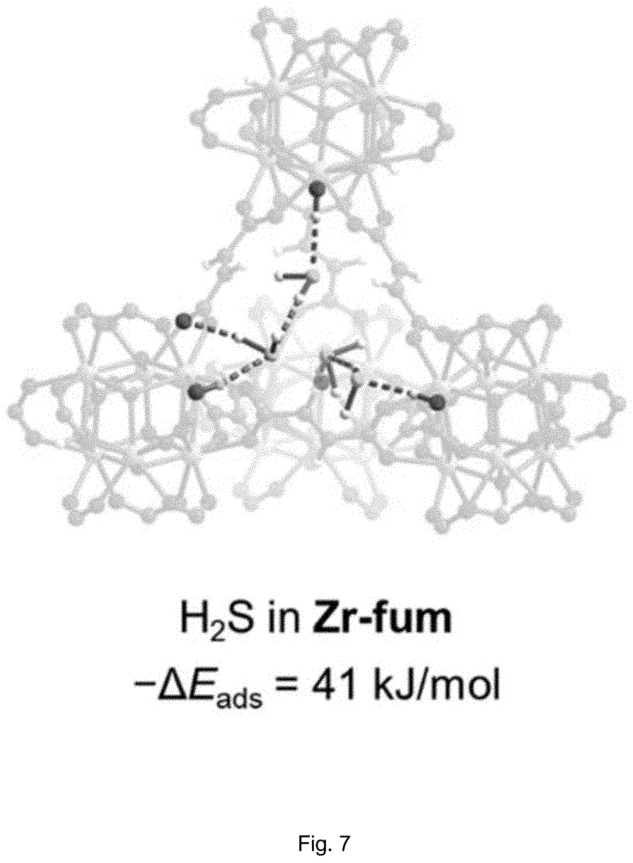

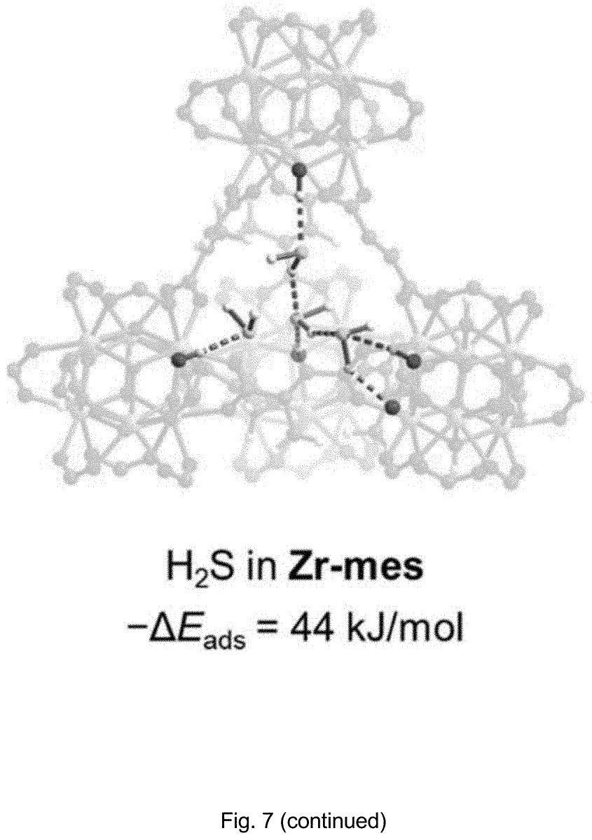

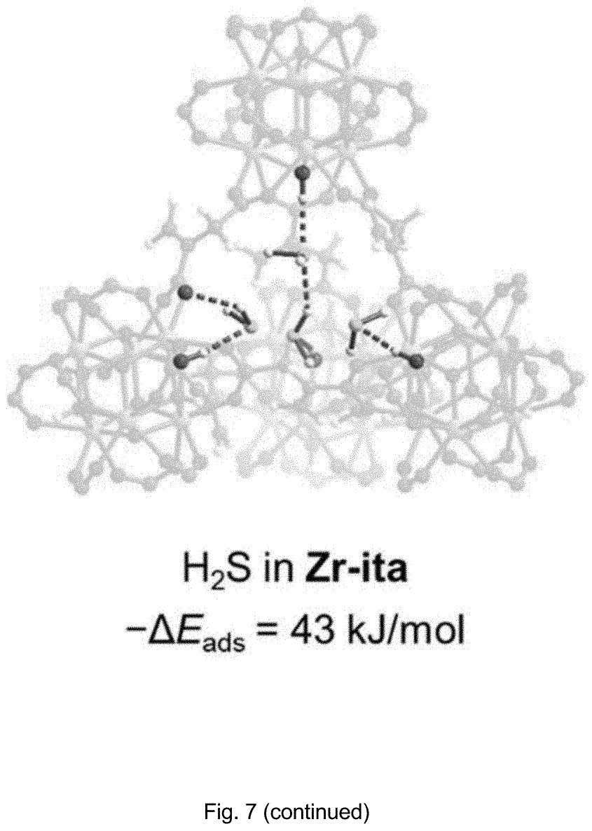

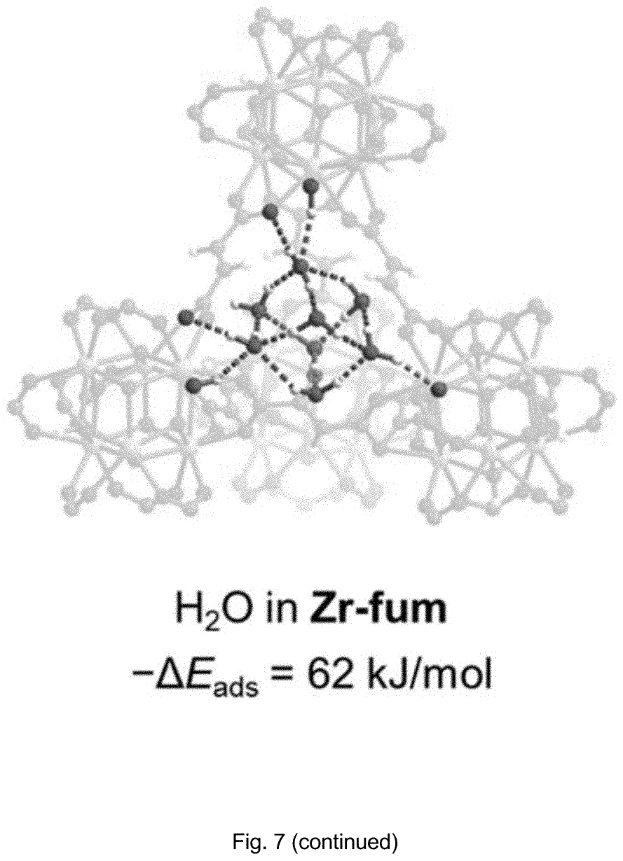

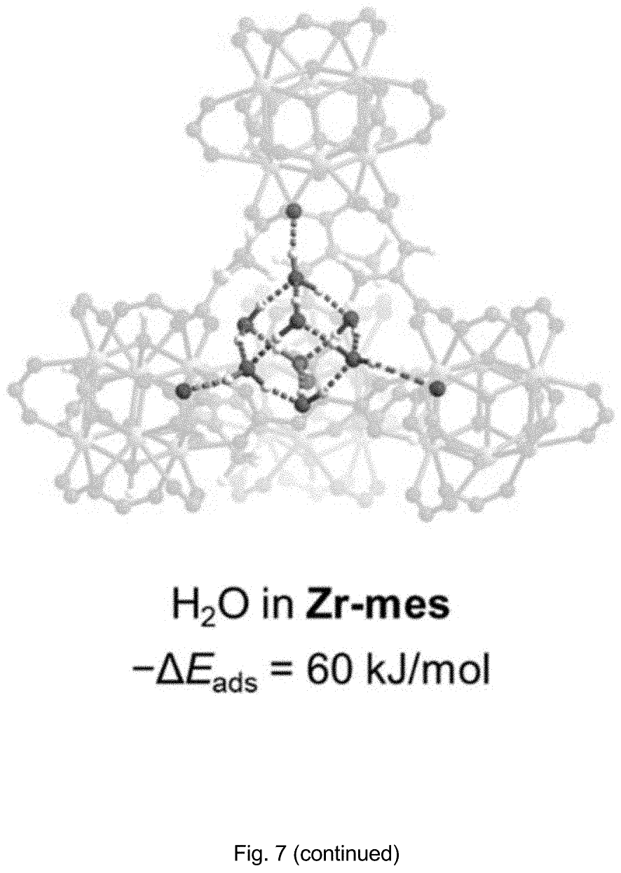

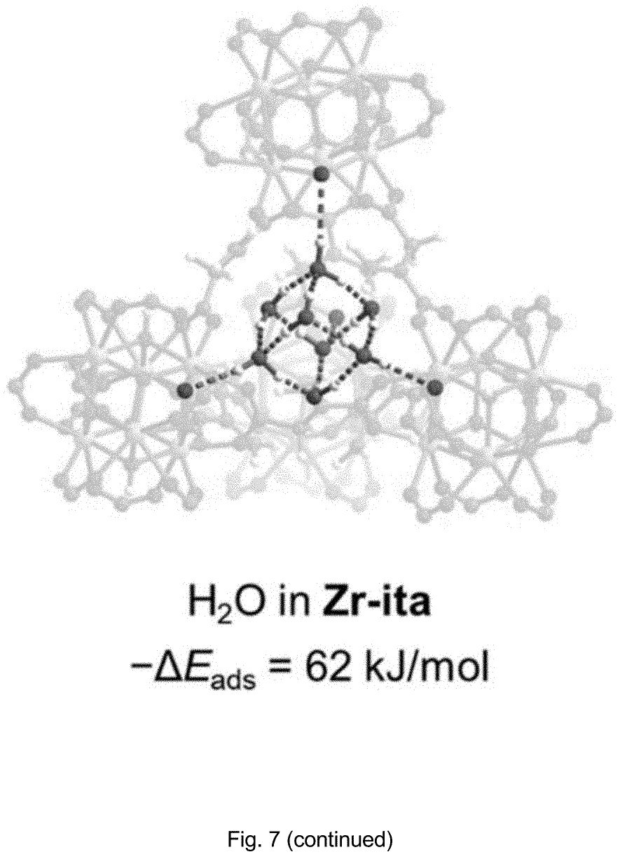

Due to the microcrystalline nature of the MOFs studied herein, we were unable to study the binding modes of H₂S within each MOF by single-crystal X-ray diffraction. Thus, we turned to density functional theory (DFT) calculations to probe the structure of H₂S bound within Zr-fum, Zr-mes, and Zr-ita (FIG. 7). Calculations were carried out using a plane-wave basis and projector augmented wave (PAW) pseudopotentials and corrected for dispersive interactions. The fidelity of our calculations was confirmed by comparing the calculated structure for Zr-fum with the previously reported single-crystal X-ray diffraction structure. As expected, H₂S was found to preferentially bind within the tetrahedral cavities of all three frameworks (FIG. 7, first three sheets). Comparing the activated and H₂S-bound structures yielded 0 K adsorption energies (−ΔH_{ads}) of 41, 44, and 43 kJ mol⁻¹ for Zr-fum, Zr-mes, and Zr-ita, respectively, which are comparable to the experimental enthalpies of adsorption (FIG. 4). The H₂S molecules are predicted to be anchored within the tetrahedral cavities by O···H/S interactions with the OH⁻groups on the Zr₆ clusters (2.16-2.43 Å in length). Additional hydrogen-bonding interactions between adjacent H₂S molecules are predicted, with S···H/S distances ranging from 2.38 Å to 2.54 Å. Notably, these distances are slightly shorter than the S···H/S distance in (H₂S)₂ (2.78 4 likely due to the increased polarization of H₂S molecules interacting with the framework pores. The favorable packing of H₂S within the tetrahedral cavities accounts for its strong yet reversible adsorption in all three frameworks. For comparison, we also predicted the preferred binding modes for H₂O in Zr-fum, Zr-mes, and Zr-ita (FIG. 7, forth thru sixth sheets). Similar to H₂S, H₂O was found to preferentially bind within the tetrahedral cavities of all three frameworks, albeit with more favorable binding energies in every case. The calculated binding energy for water in Zr-fum (−ΔH_{ads}=62 kJ mol⁻¹) is similar to the previously reported experimental enthalpy of adsorption (−ΔH_{ads}=60 kJ mol⁻¹), validating our predictions. Notably, the stronger nature of O···HO interactions compared to S···H/S interactions favors binding additional water molecules within the tetrahedral cavities, leading to a higher predicted uptake in addition to more thermodynamically favorable binding for H₂O over H₂S. The stronger overall binding of H₂O suggests that it should be able to displace H₂S from the tetrahedral cavities of MOFs, providing an avenue to trigger H₂S release under physiological conditions.

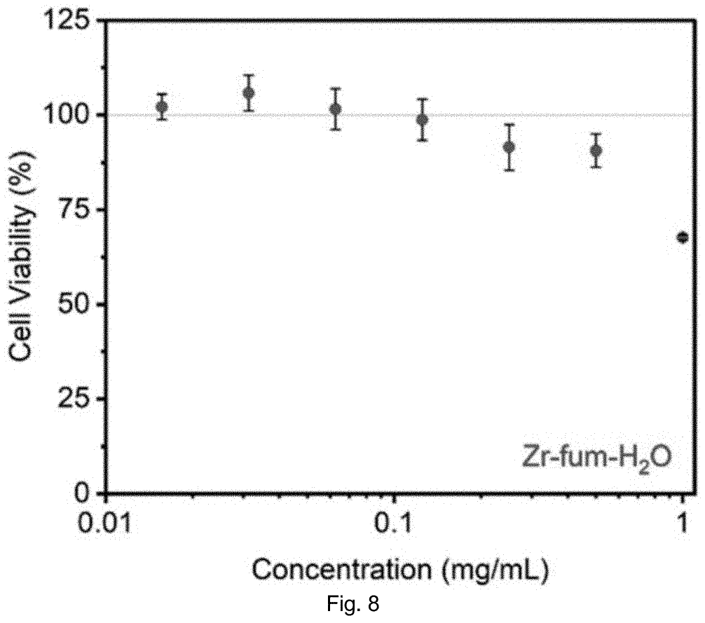

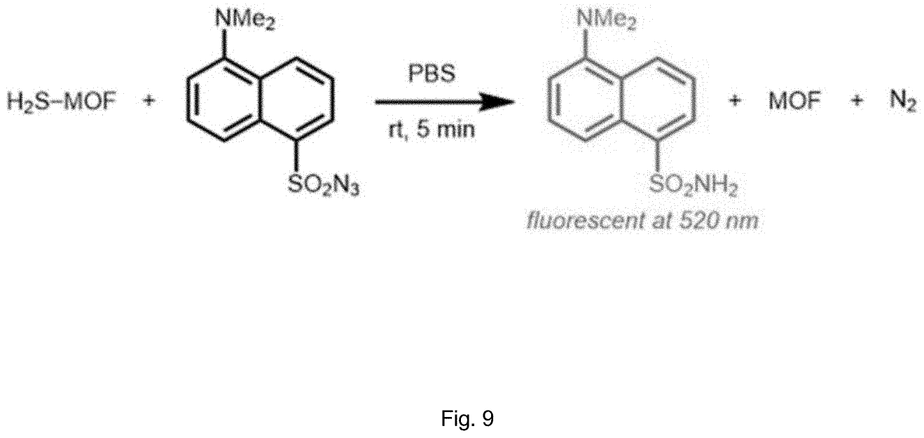

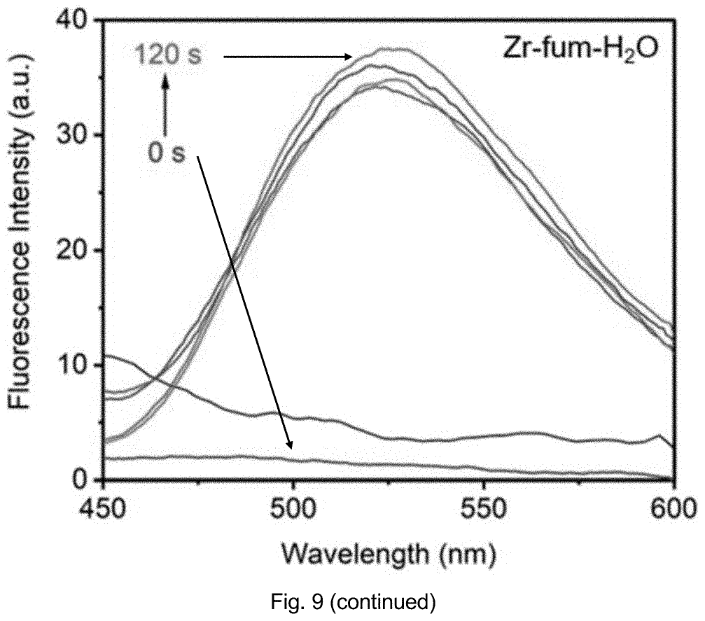

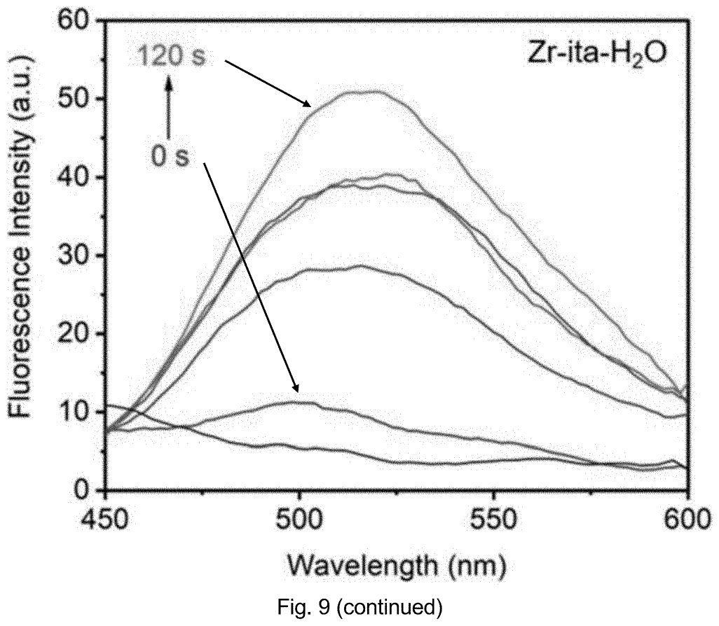

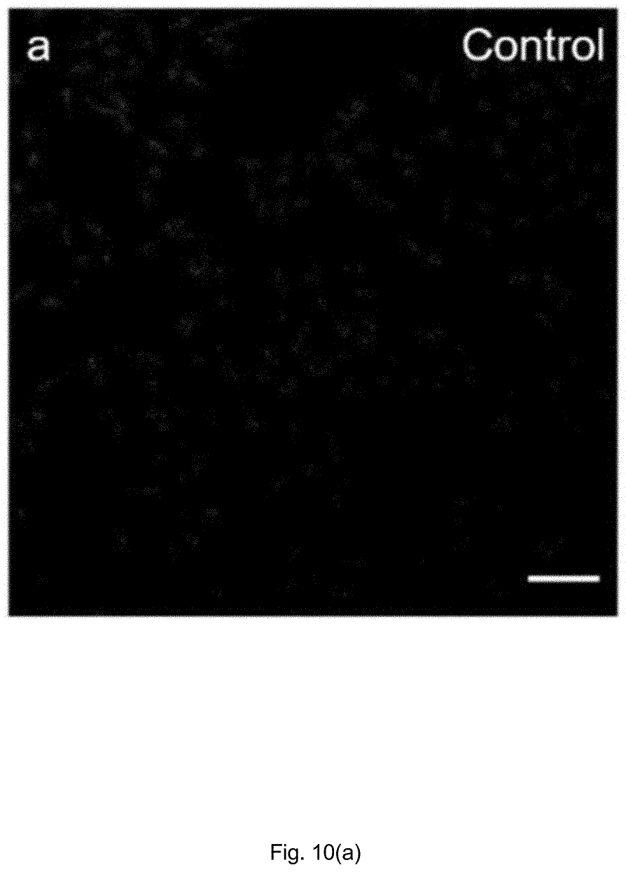

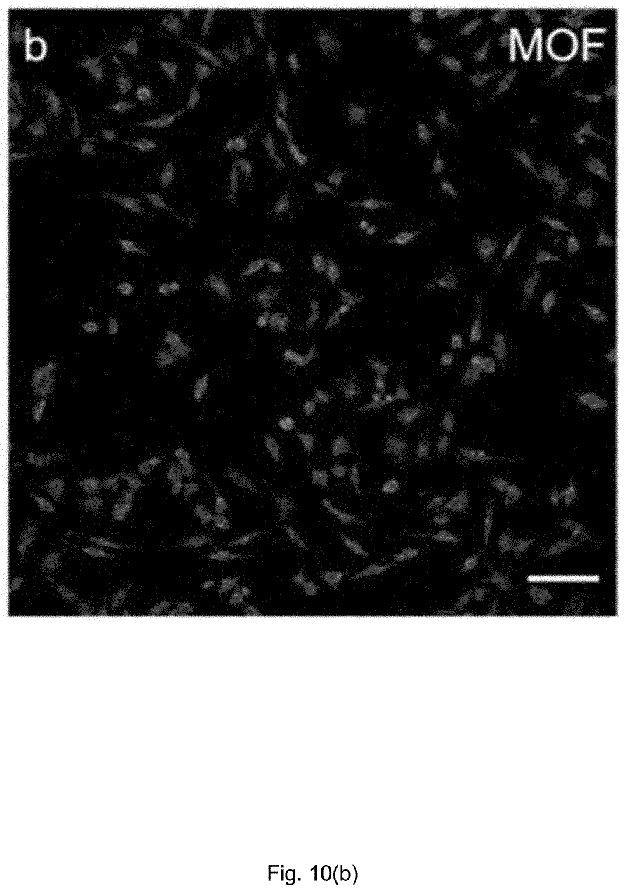

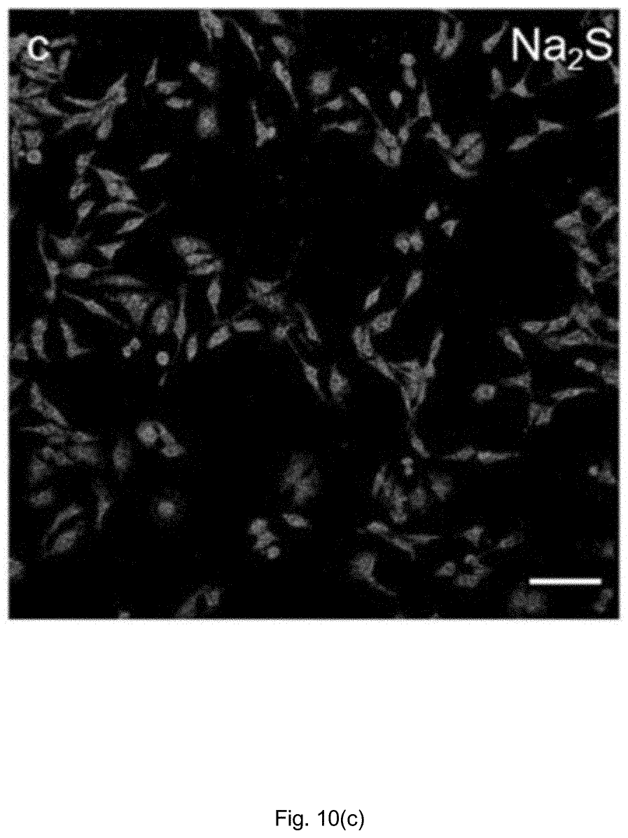

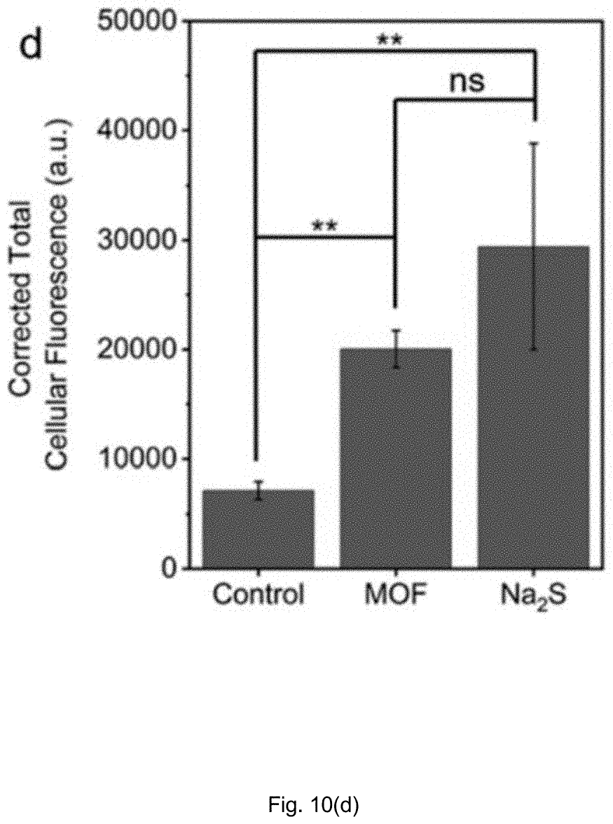

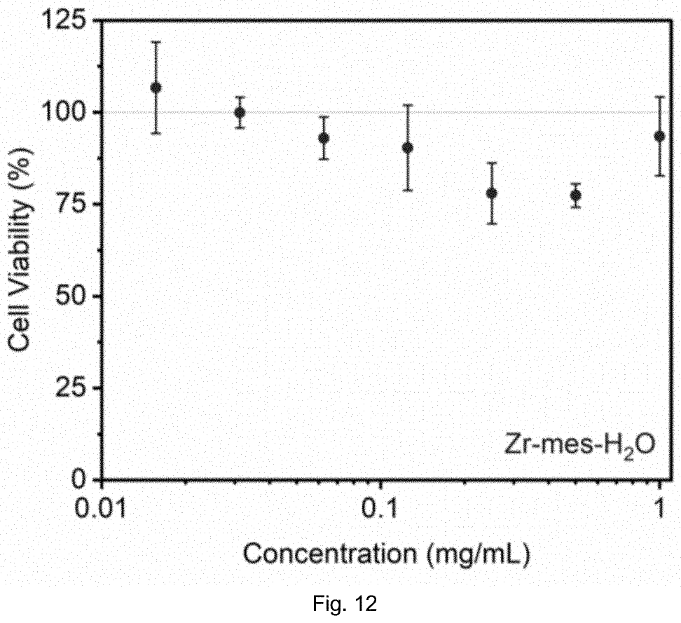

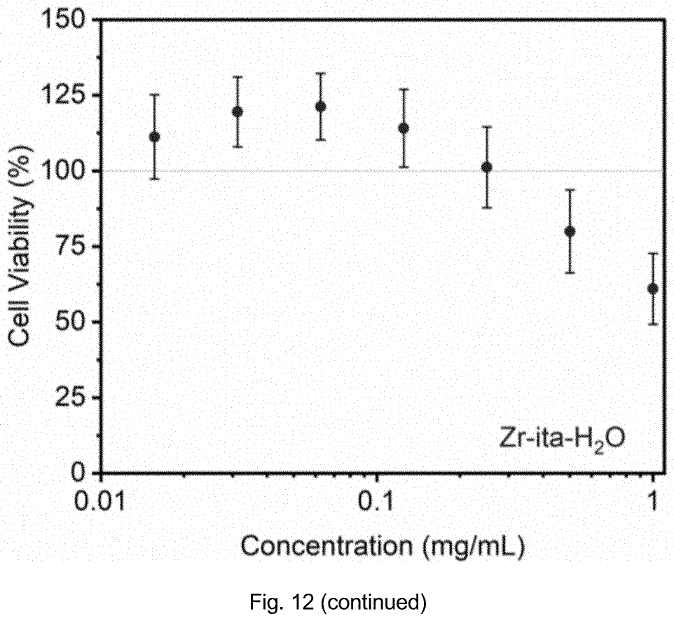

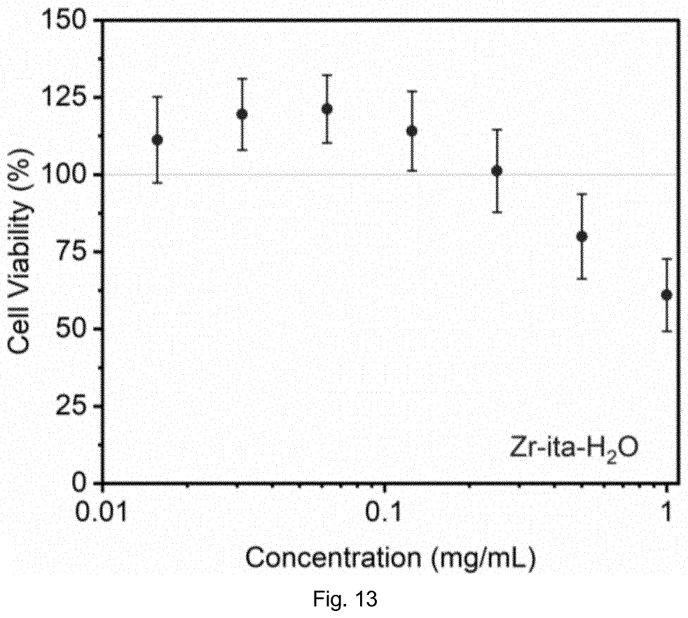

H₂S delivery from MOFs. Prior to evaluating the ability of H₂S-loaded Zr-fum, Zr-mes, and Zr-ita to release H₂S under physiologically relevant conditions, we evaluated the stability of these frameworks under aqueous conditions as well as their biocompatibility. Similar to other Zr-MOFs, all three frameworks were found to be stable in neutral deionized water (pH 7.0) and cell culture medium (pH 7.4) prepared from Dulbecco's Modified Eagle's Medium (DMEM) supplemented with 10% fetal bovine serum (FBS) over extended periods (3-10 days). Based on its high H₂S capacity and stability under physiological conditions, we selected Zr-fum-H₂O as a promising framework to study further for H₂S delivery. The biocompatibility of Zr-fum-H₂O was assessed by exposing HeLa cells to varying amounts of activated framework suspended in DMEM supplemented with 10% FBS for 72 h at 37° C. (FIG. 8). Viabilities were determined by incubating the exposed cells with (4,5-dimethylthiazol-2-yl)-2,5-diephenyltetrazolium bromide (MTT) followed by colorimetric quantification in comparison with cells that were not treated with MOF (100% viability).59 Near 100% viabilities were observed up to concentrations of 0.2 mg mL-⁻¹, and >90% viability was observed for Zr-fum-H₂O at concentrations as high as 0.5 mg mL⁻¹. This is in contrast to other MOFs such as Zn-MOF-74, which was found to be highly toxic to HeLa cells at concentrations greater than 0.1 mg mL-1. Assuming Zr-fum-H₂O is capable of delivering 4 mmol of H₂S per mg of MOF (FIG. 3), a concentration of 0.1 mg mL⁻¹ corresponds to a deliverable concentration of H₂S of up to 400 mM, which is comparable to or above endogenous concentrations (e.g., 50-160 mM in the brain). This highlights the potential benefit in using a porous solid to deliver H₂S: low concentrations (<0.1 mg mL⁻¹) of framework can be employed to release therapeutic levels of H₂S, minimizing any potential side effects from the framework itself. Promisingly, our results suggest that Zr-mes and Zr-ita are also relatively non-toxic to HeLa cells (FIGS. 12 and 13). Having established the biocompatibility of Zr-fum-H₂O, we next evaluated its ability to release H₂S upon subjection to aqueous solution (FIGS. 9 and 10). First, a known amount of H₂S dosed MOF was submerged into a large volume of phosphate buffered saline (PBS) with stirring (˜1 mg MOF per mL PBS). Aliquots from the suspension were quenched with a large excess (>20 equiv.) of the fluorescent probe 1,5-dansyl azide (DNS-az), which exhibits rapid turn-on fluorescence (wavelength max =520 nm) upon reaction with H₂S. The release of H₂S from H₂S-Zr-fum-H₂O into PBS was reproducibly confirmed by fluorescence spectroscopy (FIG. 9, second panel). The concentration of H₂S in solution reached saturation in approximately 30 seconds;

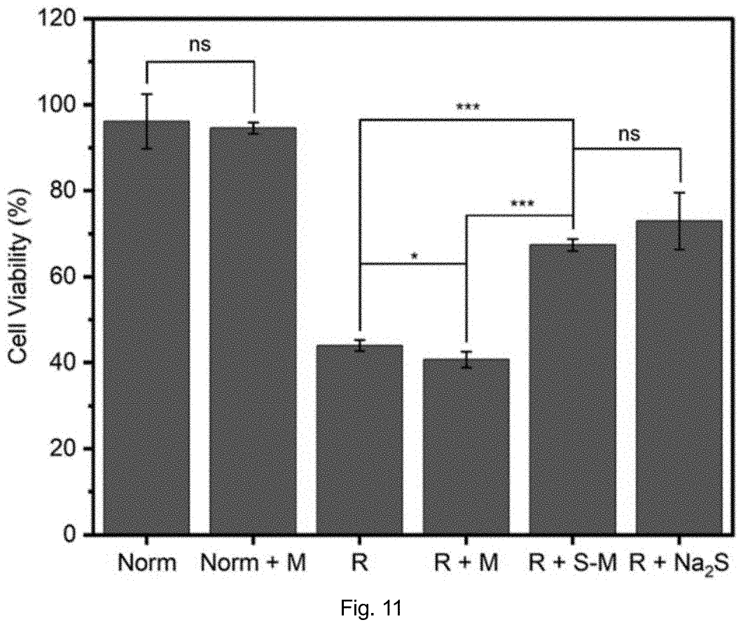

given the large excess of free DNS-az present in solution, this suggests that the release of H₂S from Zr-fum-H₂O is complete in less than 30 s. Importantly, a control experiment in which H₂S was dosed into a tube lacking MOF and then “poured” into PBS following the same procedure produced no detectable amounts of H₂S, confirming that all of the H₂S was released from Zr-fum-H₂O (black line, FIG. 9). We also reproducibly confirmed the release of H₂S from H₂S-Zr-ita-H₂O using the same assay (FIG. 9, third panel). In contrast to Zr-fum-H₂O, H₂S release from Zr-ita-H₂O took 120 s to reach saturation, indicating that H₂S release from Zr-ita-H₂S may be slower than from Zr-fum-H₂O. Unfortunately, due to unavoidable loss of gaseous H₂S to air this assay cannot be used to reliably quantify the concentration of H₂S released into solution. Notably, while the release of H₂S from Zr-fum-H₂S and Zr-ita-H₂O into PBS is relatively rapid, H₂S release under ambient conditions from all three MOFs is quite slow. As assessed by exposure to strips of filter paper soaked in lead chloride solution, all three frameworks were found to still contain H₂S after standing for at least four days in humid air, with Zr-fum-H₂S still containing H₂S after six days in humid air. In contrast, both Na₂S.9H₂O and NaSH were found to completely hydrolyze and degrade in less than 5 h under ambient conditions. The clean release of H₂S into water and the long-term stability of these MOFs in humid air makes them ideal solid sources of H₂S that can be easily handled in the laboratory. The ability of H₂S-Zr-fum-H₂O to deliver H₂S to cells under physiologically-relevant conditions was further probed using confocal fluorescence microscopy (FIG. 10). Specifically, HeLa cells were incubated first with DMEM containing WSPS (WSP=Washington State Probe), a selective fluorescence probe for intracellular ambient conditions. The clean release of H₂S into water and the long-term stability of these MOFs in humid air makes them ideal solid sources of H₂S that can be easily handled in the laboratory. The ability of H₂S-Zr-fum-H₂O to deliver H₂S to cells under physiologically-relevant conditions was further probed using confocal fluorescence microscopy (FIG. 10). Specifically, HeLa cells were incubated first with DMEM containing WSPS (WSP=Washington State Probe), a selective fluorescence probe for intracellular H₂S, followed by rinsing and exposure to water (FIG. 10a), H₂S-Zr-fum-H₂O (FIG. 10b), or Na₂S·9H₂O (FIG. 10c). For both H₂S-Zr-fum-H₂O and Na₂S·9H₂O, a statistically significant enhancement in corrected total cellular fluorescence (CTCF) was observed compared to the control experiment, validating the release and cellular uptake of H₂S in both cases. Together, these fluorescence experiments (FIGS. 9 and 10) confirm the ability of H₂S-Zr-fum-H₂O to release H₂S under physiologically relevant conditions for subsequent uptake by cells. One of the primary proposed therapeutic modes of ₂S is its ability to mediate oxidative stress within cells. For example, H₂S has been shown to limit the oxidative damage to tissue that occurs following the temporary blockage of blood flow during a stroke or myocardial infarction when a period of ischemia or hypoxia is followed by a return of normoxic blood flow, known as ischemia-reperfusion injury (R). We evaluated the ability of Zr-fum-H₂O to deliver H₂S and mitigate reperfusion injury using an in vitro model of this condition, hypoxia-reoxygenation, in H9c2 rat cardiomyoblast cells (FIG. 11). As expected, cardiomyoblast cells incubated under normoxic conditions in the absence (Norm) or presence of desolvated Zr-fum-H₂O (Norm+M) demonstrated similar viabilities (96±6% and 94±1%, respectively). The latter result is consistent with the lack of toxicity observed for this material with HeLa cells (FIG. 9). In contrast, cells incubated under hypoxic conditions (5% CO₂ in N₂ for 10 minutes) followed by a return to normoxic conditions, simulating a reperfusion injury (R), demonstrated significantly reduced viabilities (44±1%). However, subjecting cells to this reperfusion injury model in the presence of H₂S—Zr-fum-H₂O (R+S-M) led to a significant improvement in their viabilities (67±1%), confirming the potential of Zr-fum-H₂O as a delivery vessel for therapeutic H₂S. Importantly, the presence of activated MOF (R+M) did not lead to an increase in viability compared to cells in the absence of MOF (41±1%), corroborating that the therapeutic value of H₂S-Zr-fum-H₂O is likely due to H₂S release. Likewise, a statistically identical improvement in cell viabilities (73±7%) was observed using Na₂S.9H₂S in place of H₂S-Zr-fum-H₂S (R+Na₂S), confirming that H₂S-loaded MOFs provide similar therapeutic value as traditional sources of H₂S but with enhanced stability in the solid state. Overall, our results suggest that Zr-fum-H₂O, as well as Zr-mes-H₂ and Zr-ita-H₂O, represent promising vehicles for the delivery of biologically active H₂S under physiological conditions.

We demonstrated that biocompatible MOFs that are completely stable to H₂S, including Zr-fum, Zr-mes, and Zr-ita, present a promising new direction for the design of vehicles for H₂S release under physiological conditions. In particular, given its excellent stability, high H₂S capacity, lack of toxicity, ambient stability, and performance in a cellular hypoxia-reoxygenation injury assay, Zr-fum-H₂O represents a next-generation platform for the therapeutic delivery of H₂S. In addition to Zr-fum-H₂O, this work also adds Zr-mes-H₂O and Zr-ita-H₂O to the growing lexicon of biocompatible MOFs for drug delivery applications. Future work will focus on functionalizing the surface of these frameworks to further control the rate and location of H₂S delivery under physiological conditions.

General procedures. All reagents were purchased from commercial vendors and used without additional purification. The metal salts zirconyl chloride octahydrate (98%, Alfa Aesar) and zirconium (IV) chloride (99.5%, Alfa Aesar) were kept in desiccators when not in use. Fumaric acid (99%), mesaconic acid (99%), 2,5-dihydroxyterephthalic acid (H4dobdc, 95%), reagent-grade N,Ndimethylformamide (DMF), lead (II) chloride (anhydrous, 99.99%), potassium bromide (99%), sodium azide (99%), zinc nitrate hexahydrate (98%), and sodium sulfide nonahydrate (98%) were purchased from Sigma-Aldrich. Itaconic acid (99%, Alfa Aesar) was provided by the group of Prof. Brett Fors (Cornell University). Reagent-grade acetic acid was purchased from EMD Chemicals. Reagent-grade formic acid was purchased from Mallinckrodt Chemical. Absolute ethanol, acetonitrile, hexanes, methanol, dichloromethane (DCM), tetrahydrofuran (THF, 99%), tripotassium phosphate (99%), pH 3 buffer (potassium hydrogen phthalate), pH 7 buffer (monopotassium phosphate, sodium hydroxide, water), and pH 10 buffer (potassium hydroxide, potassium carbonate, potassium borate, water) solutions were purchased from Fisher Scientific or VWR. 5-(dimethylamino)naphthalene-1-sulfonyl chloride (dansyl chloride, 98%) was purchased from Cayman Chemical. Phosphate buffered saline (PBS) and fetal bovine serum (FBS) were purchased from Corning Life Sciences (Tewksbury, MA). A cylinder of hydrogen sulfide (H₂S, 99.5%) was purchased from Airgas. A solution of H₂S in THF (0.8 M) was purchased from Fluka Chemical. Deuterated dimethyl sulfoxide (DMSO-d6, 99%) and deuterium oxide (D2O, 99.9%) were purchased from Cambridge Isotope Laboratories. Infrared spectra were collected on a Bruker Tensor II IR spectrometer with a diamond Attenuated Total Reflectance (ATR) attachment. Diffuse reflectance infrared Fourier transform spectroscopy (DRIFTS) spectra were collected on a Bruker Tensor II IR spectrometer with a Harrick Praying Mantis attachment in a low temperature reaction chamber. Fluorescence spectra were measured using a Cary Eclipse fluorescence spectrophotometer (Agilent, Santa Clara, CA). Surface area data were collected on a Micromeritics 3-flex gas sorption analyzer using ultrapure N2 (99.999%) and a liquid N2 bath. Brunauer-Emmett-Teller (BET) and Langmuir surface areas were determined by linear least squares regression analysis using the linearized forms of the BET and Langmuir equations, respectively. Water and H₂S adsorption isotherms were collected on a Micromeritics 3-flex gas sorption analyzer. MOFs were activated at 100° C. or 180° C. in the case of Zn-MOF-74 for 48 h prior to any gas sorption measurements. Powder X-ray diffraction (PXRD) patterns were collected on a Rigaku Ultima IV diffractometer equipped with a CuKα source (λ=1.54 Å) and were baseline corrected after data collection. ₁H-NMR data were collected on a Bruker INOVA 500 MHz spectrometer and are referenced to residual solvent. The use of this instrument is supported by the National Science Foundation (CHE-1531632). Decomposition profiles were collected on a Q500 V6.7 thermogravimetric analyzer using a temperature ramp of 5.00° C./min from room temperature to 800.00° C. Scanning electron microscopy images were taken at 1.0 kV using a Zeiss Gemini 500 scanning electron microscope. The powder samples were immobilized on carbon tape mounted on an aluminum stub. The samples were blown using compressed air to remove excess material not stuck to the tape and then were coated with a carbon layer. X-ray photoelectron spectroscopy (XPS) spectra were collected using a Scienta Omicron ESCA-2SR with an operating pressure of 1×10-9 Torr. Monochromatic Al Kα x-rays (1486.6 eV) were generated at 300 W (15 kV; 20 mA) with photoelectrons collected from a 2 mm diameter analysis spot. Photoelectrons were collected at a 0° emission angle with a source to analyzer angle of 54.7°. A hemispherical analyzer determined the electron kinetic energy, using a pass energy of 200 eV for wide/survey scans, and 50 eV for high resolution scans. A flood gun was used for charge neutralization on all samples. Survey scans, C1s, O1s, and S2p high resolution scans were collected for all samples. Zn2p and Zr3d high resolution scans were collected for Zn-MOF-74 and Zr-fum samples, respectively. Spectra were analyzed in CasaXPS; each scan was binding energy calibrated to the C1s primary peak position set to 284.8 eV. H₂S adsorption data were fit using the Dual-Site Langmuir-Freundlich model (Eq. 2), where Q(P) is the predicted uptake Q at pressure P in mmol/g, Qsati is the saturation pressure of binding site i in mmol/g, bi is the Langmuir parameter of site i, vi is the Freundlich parameter of site i, —Si is the entropy of binding site i in J/mol·K, R is the ideal gas constant, Eᵢ is the enthalpy of adsorption for binding site i in kJ/mol, and T is the temperature in K. The isotherms were fit with either v1 and v2 set as 1 (Dual-Site Langmuir model) or with v1 and v2 allowed to vary freely (Langmuir-Freundlich model). Fits were obtained using Solver in Microsoft Excel.

$$Q ( P ) = \frac{{{Q}_{s a t 1} ( {b}_{1} P )}^{{v}_{1}}}{1 + {( {b}_{1} P )}^{{v}_{1}}} + \frac{{{Q}_{s a t 2} ( {b}_{2} P )}^{{v}_{2}}}{1 + {( {b}_{2} P )}^{{v}_{2}}} ; {b}_{i} = {e}^{( \frac{- {S}_{i}}{R} ) ( \frac{1000 \cdot {E}_{i}}{R T} )} \qquad Eq . 2$$

Heats of adsorption were calculated using the Clausius-Clapeyron equation (Eq. 3), where PQ are pressure values corresponding to the same loading Q, −ΔH_{ads} is the differential enthalpy of adsorption in kJ/mol, R is the ideal gas constant, T is the temperature in K, and c is a constant. Fits over a range of Q values were obtained using Mathematica.

$$ln ( {P}_{Q} ) = ( \frac{Δ {H}_{ads}}{R} ) ( \frac{1}{T} ) + c \qquad Eq . 3$$

Small-scale preparations of MOFs under aqueous conditions with formic acid as modulator. Zr-fum-H₂O. Fumaric acid (58.0 mg, 0.50 mmol, 1.00 eq) and ZrOCl₂·8H₂O (161 mg, 0.50 mmol, 1.00 eq) were added to a 15 mL screw cap reaction tube along with deionized water (2 mL). Next, either 0 eq, 10 eq (189 μL, 5.00 mmol), 30 eq (566 μL, 15.0 mmol), 50 eq (943 μL, 25.0 mmol) or 80 eq (1.51 mL, 40.0 mmol) of formic acid were added. The tube was heated to 95° C. in an aluminum heating block without stirring for 24 h. After removing the tube from the heating block and allowing it to cool to room temperature, the remaining solvent was pipetted off of the settled precipitate and replaced with fresh ethanol (10 mL). The ethanol was replaced once every 24 h for three days. Powder X-ray diffraction patterns were collected after the last ethanol soak.

Zr-mes-H₂O. Mesaconic acid (65.0 mg, 0.50 mmol, 1.00 eq) and ZrOCl₂·8H₂O (161 mg, 0.50 mmol, 1.00 eq) were added to a 15 mL screw cap reaction tube along with deionized water (2 mL). Next, either 0 eq, 30 eq (566 μL, 15.0 mmol), 50 eq (943 μL, 25.0 mmol) or 80 eq (1.51 mL, 40.0 mmol) of formic acid were added. The tube was heated to 95° C. in an aluminum heating block without stirring for 24 h. After removing the tube from the heating block, the remaining solvent was pipetted off of the settled precipitate and replaced with fresh ethanol (10 mL). The ethanol was replaced once every 24 h for three days. Powder X-ray diffraction patterns were collected after the last soak.

Preparation and characterization of Zr₆O₄(OH)₄(fumarate)₆, Zr-fum-DMF. This procedure was adapted from the literature. Fumaric acid (1.16 g, 10.0 mmol, 1.00 eq), ZrOCl₂·8H₂O (3.24 g, 10.0 mmol, 1.00 eq), and formic acid (14.0 mL, 371 mmol, 37.0 eq) were added to a screw cap, high pressure reaction vessel equipped with a stir bar. Next, DMF (40 mL) was added, and the reaction vessel was sealed. The mixture was stirred and heated at 120° C. for 24 h. The heterogeneous reaction mixture was allowed to cool to room temperature and filtered.

The resulting precipitate was transferred into a screw cap jar filled with DMF (80 mL), and the mixture was allowed to stand for 24 h at room temperature. The DMF was then decanted and replaced with the same volume of fresh DMF. This procedure was repeated an additional two times for a total of three DMF soaks, at which point the DMF was replaced with ethanol (80 mL), and the process repeated another three times for a total of six washes. The solid was then activated under high vacuum (<100 mbar) at 100° C. overnight to yield Zr₆O4(OH)₄(fumarate)₆ as a white solid.

Preparation and characterization of aqueous Zr₆O₄(OH)₄(fumarate)₆, Zr-fum-H₂O. Fumaric acid (1.16 g, 10.0 mmol, 1.00 eq), ZrOCl₂·8H₂O (3.24 g, 10.0 mmol, 1.00 eq), and formic acid (11.3 mL, 300 mmol, 30.0 eq) were added to a screw cap, high pressure reaction vessel equipped with a stir bar. Next, deionized water (40 mL) was added, and the reaction vessel was sealed. The mixture was stirred and heated at 95° C. for 24 h. The heterogeneous reaction mixture was allowed to cool to room temperature. After cooling, the mixture was transferred into a centrifuge tube and centrifuged at 4000 rpm for 10 minutes two times. The solvent was decanted, and the resulting precipitate was transferred into a screw cap jar filled with ethanol (80 mL). The solid was allowed to stand for 24 h at room temperature. The mixture was centrifuged as detailed above. The ethanol was then decanted and replaced with the same volume of fresh ethanol. This procedure was repeated a total of three times. The solid was then activated under high vacuum (<100 mbar) at 100° C. overnight to yield Zr₆O₄(OH)₄(fumarate)₆ as a white 20 solid.

Preparation and characterization of Zr₆O₄(OH)₄(mesaconate)₆, Zr-mes-DMF. Mesaconic acid (1.30 g, 10.0 mmol, 1.00 eq), ZrOCl₂·8H₂O (3.24 g, 10.0 mmol, 1.00 eq), and formic acid (14.0 mL, 371 mmol, 37.0 eq) were added to a screw cap, high pressure reaction vessel equipped with a stir bar. Next, DMF (40 mL) was added, and the reaction vessel was sealed. The mixture was stirred and heated at 120° C. for 24 h. The heterogeneous reaction mixture was allowed to cool to room temperature and filtered. The resulting precipitate was transferred into a screw cap jar filled with DMF (80 mL), and the mixture was allowed to stand for 24 h at room temperature. The DMF was then decanted and replaced with the same volume of fresh DMF. This procedure was repeated an additional two times for a total of three DMF soaks, at which point the DMF was replaced with ethanol (80 mL) and the process repeated another three times for a total of six washes. The solid was then activated under high vacuum (<100 mbar) at 100° C. overnight to yield Zr₆O₄(OH)₄(mesaconate)₆ as a fine white powder.

Preparation and characterization of aqueous Zr6O4(OH)4(mesaconate)6, Zr-rmes-H₂O. Mesaconic acid (1.30 g, 10.0 mmol, 1.00 eq), ZrOCl₂·8H₂O (3.24 g, 10.0 mmol, 1.00 eq), and formic acid (11.3 mL, 300 mmol, 30.0 eq) were added to a screw cap, high pressure reaction vessel equipped with a stir bar. Next, deionized water (40 mL) was added, and the reaction vessel was sealed. The mixture was stirred and heated at 95° C. for 24 h. The heterogeneous reaction mixture was allowed to cool to room temperature and filtered. The precipitate was transferred into a screw cap jar filled with ethanol (80 mL), and the mixture was allowed to stand for 24 h at room temperature. The ethanol was then decanted and replaced with the same volume of fresh ethanol. This procedure was repeated a total of three times. The solid was then activated under high vacuum (<100 mbar) at 100° C. overnight to yield Zr₆O₄(OH)4(mesaconate)₆ as a fine white powder.

Preparation and characterization of Zr6O4(OH)4(itaconate)₆, Zr-ita-DMF. Various acid modulators including acetic acid, L-proline, HC1, pivalic acid, and formic acid were used in an attempt to produce crystalline Zr-ita-DMF. However, in all cases amorphous solids were produced (not shown). Thus, a large-scale batch was prepared with no acid modulator for further characterization. Itaconic acid (0.98 g, 7.50 mmol, 1.00 eq) and ZrCl4 (1.75 g, 7.50 mmol, 1.00 eq) were added to a screw cap, high pressure reaction vessel equipped with a stir bar. Next, DMF (150 mL) and deionized water (0.40 mL, 22.5 mmol, 3.00 eq) were added, and the reaction vessel was sealed. The mixture was stirred and heated at 120° C. for 24 h. The heterogeneous reaction mixture was allowed to cool to room temperature and filtered. The precipitate was transferred into a screw cap jar filled with DMF (80 mL), and the mixture was allowed to stand for 24 h at room temperature. The DMF was then decanted and replaced with the same volume of fresh DMF. This procedure was repeated an additional two times for a total of three DMF soaks, at which point the DMF was replaced with THF (80 mL) and the process repeated another three times for a total of six washes. The solid was then activated under high vacuum (<100 mbar) at 100° C. overnight to yield Zr6O4(OH)₄(itaconate)₆ as a white solid.

Characterization of MOFs prepared under aqueous conditions exposed to H₂S solution. General procedure. In a nitrogen-filled glovebox, a 15 mL screw cap reaction tube was loaded with desolvated MOF (˜50 mg) and capped. The tube was removed from the glovebox and placed under a dry atmosphere of nitrogen. Next, H₂S in THF (0.8 M, 1.0 mL) was added via syringe under a constant stream of nitrogen. The cap was replaced with one that had not been punctured, and the tube was left to stand for 48 h at 50° C. The MOFs were collected by filtration and washed with THF into a filter flask containing bleach to quench any remaining H₂S. The solids were then characterized.

Delivery of H₂S under biologically relevant conditions. Serum and DI water stabilities. A 5 mL scintillation vial was loaded with desolvated MOF (˜50 mg) and either serum or DI water (3.0 mL) was added via syringe. The vial was capped, and the tube was left to stand for either 3 days (serum) or 10 days (DI water) at room temperature. The serum measurements were carried out using at Dulbecco's Modified Eagle's Medium (DMEM) supplemented with 10% fetal bovine serum (FBS), which is the standard medium used for all cell culture experiments in this work. The MOFs were collected by filtration and washed with deionized water. The solids were then characterized. Together, these results suggest that the MOFs would likely undergo slow degradation upon submersion in serum for extended periods of time, but they are stable enough for delivery of H₂S.

Synthesis of 1,5-dansyl azide (DNS-az). This compound was synthesized by a modified literature procedure. A solution of dansyl chloride (250 mg, 0.93 mmol) in ethanol (15 mL) was added dropwise to a rapidly stirred solution of sodium azide (100 mg, 1.54 mmol) in 50% aqueous ethanol (7 mL). The resulting yellow solution was stirred for 4 h, at which time the solvent was removed by rotary evaporation to yield the crude product as a yellow oil. The oil was purified by silica gel chromatography (2:1 hexanes/CH₂l₂). The pure fractions were pooled and evaporated to yield DNS-az as a bright yellow oil, which was dried under high vacuum overnight. Yield: 190 mg (73.9%). 41 NMR (500 MHz, DMSO-d₆): 67 5 8.66 (d, J=10 Hz, 1H), 8.37 (d, J=10 Hz, 1H), 8.03 (d, J=10 Hz, 1H), 7.74 (m, 2H), 7.34 (d, J=10 Hz, 1H), 2.85 (s, 6H). ¹³C NMR (125 MHz, DMSO-d₆): δ 151.97, 132.72, 130.30, 129.60, 129.17, 128.72, 123.75, 117.62, 115.92, 45.06. These spectra are consistent with those reported in the literature.

H₂S detection via fluorescence spectroscopy. The turn-on fluorescent probe 1,5-dansyl azide (DNS-az) was used to detect H₂S in solution. A known amount of desolvated Zr-ita-H₂O or Zr-fum-H₂O (50-100 mg) was weighed and activated for 48 h at 100 ° C. under vacuum 30 (<10 μbar). The MOF was then dosed with ˜1 bar of H₂S at 30° C. and allowed to equilibrate until <0.01% change in the H₂S pressure occurred over a 45 s interval (approximately 3-4 h in each case). The exact amount of H₂S adsorbed was recorded. At the time of the experiment, the tube with MOF was purged with N₂ for 90 s to remove gaseous H₂S before the sample was poured into a stirred solution of phosphate buffered saline (PBS; pH 7.4) at a typical concentration of ˜1 mg MOF/mL of PBS. Assuming a capacity of 4 mmol/g of H₂S in Zr-fum-H₂O, this corresponds to a maximum concentration of 4 mM for H₂S in the PBS solution, if all of the H₂S was released by the MOF into solution. A control experiment in which a tube lacking MOF was dosed with H₂S, purged with N₂ for 90 s, and then “poured” into PBS yielded no detectable amounts of H₂S in solution, confirming that this purging procedure is sufficient to remove gaseous H₂S from the tube. At 30 second intervals, 300 μL aliquots of the suspension (corresponding to a maximum amount of 1.2 μmol of H₂S) was removed (taking care not to remove any solids; a syringe filter was used in the case of Zr-fum-H₂) and added to a freshly-prepared solution of DNS-az (25 μmol) in CH₃CN (1 mL, final concentration of 25 mM). The large excess of DNS-az (25 μmol) compared to H₂S (maximum of 1.2 μmol for Zr-fum-H₂O) in the aliquot guarantees that the DNS-az probe should not be saturated during the experiment. The solution was allowed to incubate at 23° C. for 5 min (min=minute(s)) before the fluorescence of the probe was measured at 520 nm (with excitation at 340 nm). Due to unavoidable loss of gaseous H₂S to air, this assay cannot be used to reliably quantify the amount of H₂S released into solution. However, the kinetics of H₂S release were found to be relatively reproducible between samples.

Cell lines and culture conditions. HeLa and H9c2 cells were obtained from American Type Culture Collection (ATCC, Washington D.C.) and cultured as adherent monolayers in a humidified 5% CO₂ atmosphere at 37° C. in DMEM supplemented with 10% FBS. Cells were checked for contamination monthly using the PlasmoTest mycoplasma detection kit from InvivoGen (San Diego, CA). Cytotoxicity assay. HeLa or H9c2 cells were seeded in 96-well plates with 2000 cells/well and allowed to reattach overnight. The following day, the culture media was removed, and cells were treated with suspensions containing varying concentrations of the desired MOF in in DMEM supplemented with 10% FBS and incubated for 72 h at 37° C. Following treatment, the cells were incubated in DMEM containing 1 mg/mL (4,5-dimethylthiazol-2-yl)-2,5-diephenyltetrazolium bromide (MTT) without FBS for 3 hours. Following incubation, the media was removed, and the purple formazan crystals were solubilized using 150 μL of an 8/1 S65 DMSO/glyceine buffer (pH 10) mixture. The absorbance at 570 nm of each well was measured using a BioTek Synergy HT plate reader. Results are reported as the average cell viability of 6 wells/concentration compared to untreated cells from three independent trials, with the standard deviation (SD) reported as the error (±SD).

Cellular hypoxia-reoxygenation model of ischemia-reperfusion injury. On the day before the experiment, H9c2 rat cardiomyoblast cells were seeded in a 96 well plate at a density of 8000 cells/well and incubated at 37° C. overnight. To initiate the experiment, the culture media was removed, and the cells were washed with 100 μL of PBS. The assay was initiated by placing the cells in 200 μL of modified Gey's balanced salt solution (GBSS) containing 120 mM NaCl, 5 mM KCl, 1.5 mM CaCl2, 0.28 mM MgSO₄, 1 mM MgCl₂, 0.840 mM Na₂HPO₄, 0.220 mM KH₂PO₄, and 27 mM NaHCO₃. The plates were then placed in a hypoxia chamber and flushed with 5% CO₂ in N₂ for 10 minutes. The chamber was sealed, and the cells were incubated under hypoxic conditions for 2.5 h at 37° C. Following hypoxia, the cells were incubated under normoxic conditions in DMEM containing 10% FBS and the H₂S-loaded Zr-fum-H₂O at a concentration of 0.05 mg/mL for 24 h prior to measuring the viability using the MTT assay as described above. Viability is reported relative to control cells that were incubated under normal culture conditions throughout the experiment. Another set of control cells were incubated with the MOF without previous incubation under hypoxic conditions. Results are reported as the average viability from 12 wells of a 96 well plate from three independent trials, with the standard deviation (SD) reported as the error (±SD) (ns=not significant, ***p<0.005).

H₂S Imaging in HeLa cells with WSPS. Approximately 1×105 HeLa cells were seeded in 35 mm glass-bottomed dishes (MatTek Life Sciences, Ashland, MA) and incubated overnight at 37° C. The following day, the media was removed and the cells were treated with DMEM without FBS containing 1 mM cetyl trimethyl ammonium bromide (CTAB) and 15 μM WSPS (Cayman Chemicals, Ann Arbor, MI; WSP=Washington State Probe) and incubated for 30 min at 37° C. in the dark. The dye-containing media was removed, and the cell layer was washed with PBS (2×1 mL) before treatment with either 0.05 mg/mL of H₂S-loaded Zr-fum-H₂O or 200 μM Na₂S in DMEM without FBS. The cells were then incubated for 1 h at 37 ° C. At the time of imaging, the media was removed, and the cells were washed with 1 mL PBS. Fluorescence images were collected in PBS using a Zeiss LSM710 confocal fluorescence microscope fitted with a 10×water objective with an excitation wavelength of 514 nm and an emission window of 535-657 nm. Images were analyzed and quantified using ImageJ (NIH) and the corrected total cellular fluorescence (CTCF) was calculated using the following formula: CTCF=Integrated density−(area of cell×mean fluorescence of the background) The average of 8 individual cells was used to determine the average CTCF for each replicate. Results are reported as the average of three biological replicates. Qualitative monitoring of H₂S release via lead test strips. The formation of lead sulfide on lead chloride-soaked filter paper was used to visually confirm H₂S release from Zr-mes-H₂O, Zr-fum-H₂, and Zr-ita-H₂O under ambient conditions. Filter paper cut into thin strips was dipped into a 1 wt % aqueous lead (II) chloride solution and dried for ten minutes. Once dried, the strips were added to a 25 mL scintillation vial containing ˜100 mg of H₂S-loaded MOF prepared under the same conditions as described above. Black or orange lead sulfide precipitation on the strips resulted immediately upon contact with the MOF and/or vial glass. The vials were capped, and the old strips removed and replaced once per day until they no longer turned black or orange. Images were taken of the vials each day. As a control, the same assay was used to monitor the release of H₂Sfrom a similar amount of Na₂S·9H₂O and NaSH. Owing to the rapid hydrolysis of both salts (complete degradation/dissolution within 5 h). the lead strip was not changed through the duration of the assay

Computational and structural details. To elucidate the H₂O and H₂S adsorption mechanisms, we performed first-principles density functional theory (DFT) calculations using a plane-wave basis and projector augmented-wave (PAW) pseudopotentials with the Vienna ab-initio Simulation Package (VASP) code. To include the effect of the van der Waals (vdW) dispersive interactions on binding energies, we performed structural relaxations with Grimme's D3 correction as implemented in VASP. For all calculations, we used (i) a Γ-point sampling of the Brillouin zone and (ii) a 600 eV plane-wave cutoff energy. We explicitly treated twelve valence electrons for Zr (4s²4p⁶4d²5s²), six for S (3s²3p⁴), six for O (2s²2p⁴), four for C (2s²2p²), and one for H (1s¹). All structural relaxations were performed with a Gaussian smearing of 0.05 eV. The ions were relaxed until the Hellmann-Feynman forces were less than 0.01 eVA^{-1.} Using above input parameters, we relaxed both internal coordinates and volumes. To compute the H₂O and H₂S binding energies, we optimized MOFs prior to H₂O and H₂S adsorptions (E_{MOF}), interacting with ₂O and H₂S in the gas phase (E_{H2O|H2S}) within a 20 Å×20 Å×20 Å cubic supercell, and MOF with adsorbed H₂O and H₂S molecules (E_{H2O|H2S-MOF}) using a rigid MOF. The binding energies (E_{B}) were obtained via the difference E_{B}=E_{H2O|H2S-MOF}−(E_{MOF}+E_{H2O|H2S}). The idealized Zr₆O₄(OH)₄(RCO₂)₆ clusters of Zr-fum, Zr-mes, and Zr-ita contain a mixture of four OH— and four O₂-sites to achieve charge neutrality. Initially, we computationally generated these structures without explicitly treating the protons on the Zr₆O₈ clusters for simplicity. The calculated structure of Zr-fum shows excellent agreement with the previously reported single-crystal X-ray diffraction structure of MOF-801.

H₂S and aqueous stability studies with Zn-MOF-74. Preparation of Zn-MOF-74. This procedure is adapted from the literature. A 350 mL, screw-cap high pressure reaction vessel equipped with a stir bar was charged with Zn(NO₃)₂·6H₂O (2.23 g, 7.50 mmol, 3.00 eq.), H₄dobdc (495 mg, 2.50 mmol, 1.00 eq.), fresh DMF (125 mL), and ethanol (125 mL). The mixture was sonicated until all of the solids dissolved. The reaction mixture was vigorously sparged with N₂ for 1 h. The reaction vessel was sealed, and the reaction mixture was allowed to stir slowly at 120° C. for 14 h, resulting in precipitation of a yellow powder from solution. The reaction mixture was cooled to room temperature and filtered. The collected solid was quickly transferred to a 500 mL Pyrex jar filled with DMF (250 mL). The jar was placed in an oven that had been pre-heated to 120° C. and left to stand for 24 h, after which time the nonhomogeneous mixture was filtered. The collected solid was returned to the jar with fresh DMF (250 mL and returned to an oven that had been pre-heated to 120° C. This soaking process was repeated two more times. The mixture was then filtered and transferred to a 500 mL Pyrex jar filled with methanol (250 mL), and the jar was placed in an oven that had been pre-heated to 60° C. and left to stand 24 h, after which time the non-homogeneous mixture was filtered. The collected solid was returned to the jar with fresh methanol (250 mL) and returned to an oven that had been pre-heated to 60° C. This soaking process was repeated two more times. The mixture was filtered a final time, and the collected solid was quickly transferred to a Schlenk flask under N₂. The material was activated under flowing N₂ at 180° C. for 24 h, and then by heating under high vacuum at 180° C. for 24 h.

DRIFTS measurements. To measure the DRIFTS spectrum of free H₂S gas, the sample cup of the Harrick low temperature reaction chamber was filled with KBr and evacuated to <10 mTorr before dosing with H₂S (400 mbar). To measure the DRIFTS spectrum of Zr-fum-H₂O and H₂S-dosed Zr-fum-H₂O, the sample cup of the Harrick low temperature reaction chamber was filled with a 1:5 mixture of Zr-fum-H₂O and KBr. The mixture was then activated at 100° C. for 18 hours using a Harrick ATK-024-3 temperature controller under high vacuum (<10 mTorr). The reaction chamber was subsequently pulsed with H₂S until desired pressures of 500, 750 and 1000 mbar were reached before immediately closing the chamber and measuring IR spectra.

TABLE 1
BET and Langmuir surface areas calculated
from the N₂ adsorption isotherms.

| | BET surface area | Langmuir surface area |
|---|---|---|
| MOF | (m₂/g) | (m₂/g) |
| Zr-fum-DMF | 988 ± 1 | 1322 ± 2 |
| Zr-fum-H₂O | 873 ± 2 | 1196 ± 9 |
| Zr-mes-DMF | 583 ± 1 | 663 ± 2 |
| Zr-mes-H₂O | 688 ± 1 | 830 ± 4 |
| Zr-ita-DMF | 33 | 81 ± 6 |
| Zr-ita-H₂O | 235 ± 2 | 487 ± 22 |

Although the present disclosure has been described with respect to one or more particular embodiments and/or examples, it will be understood that other embodiments and/or examples of the present disclosure may be made without departing from the scope of the present disclosure.

1. A Zr-based metal-organic framework (Zr-MOF) comprising the following formula: Zr₆O₄(OH)₄(polycarboxylate)₆, with the proviso the Zr-MOF is not Zr₆O₄(OH)₄(fumarate)₆ (Zr-fum) or Zr₆O₄(OH)₄(mesaconate)₆ (Zr-mes).

2. The Zr-MOF of claim 1, wherein the polycarboxylate is independently at each occurrence group chosen from saturated polycarboxylate groups, unsaturated polycarboxylate groups, straight-chain polycarboxylate groups, branched polycarboxylate groups, cyclic polycarboxylate groups, heterocyclic polycarboxylate groups, aromatic polycarboxylate groups, and heteroaromatic polycarboxylate groups, and any combination thereof.

3. The Zr-MOF of claim 1, wherein the polycarboxylate is independently at each occurrence chosen from carboxylate group of fumaric acid, mesaconic acid, itaconic acid, terephthalic acid, succinic acid, glutamic acid, oxalic acid, glutaric acid, 4,4′-biphenyldicarboxylic acid, 2,6-naphthalenedicarboxylic acid, trimesic acid, and tetrakis(4-carboxyphenyl)porphyrin.

4. The Zr-MOF of claim 1, wherein the Zr-MOF is completely amorphous, at least partially amorphous and/or crystalline, or completely crystalline.

5. A Zr-based MOF of claim 1, wherein the Zr-MOF comprises a plurality of pores, wherein the pores have a longest linear dimension perpendicular to the long axis of the pores of from about 0.1 Angstrom(s) (Å) to about 200 Å.

6. The Zr-MOF of claim 1, wherein the Zr-MOF comprises or exhibits one or more or all of the following:
a Brunauer-Emmett-Teller (BET) Surface Area of from about 0 square-meter(s)/gram (m²/g) to about 1000 m²/g;
a Langmuir Surface Area of 0 m²/g to about 1400 m²/g;
an Enthalpy of Adsorption (−ΔH_{ads}) for hydrogen sulfide (H₂S) of from about 0 kilojoule(s) (kJ)/mol to about 60 kJ/mol;
a H₂S uptake capacity of from about 0 millimol H₂S/gram of Zr-MOF (mmol H₂S/g Zr-MOF) to about 10 mmol H₂S/g Zr-MOF;
a H₂S release rate of from about less than 30 seconds to less than about 24 hours; or
a H₂S cycling capacity decrease of from about 0% to about 8%.

7. A method of making one or more Zr-based metal-organic framework(s) (Zr-MOF(s)) independently comprising the following formula:
Zr₆O₄(OH)₄(polycarboxylate)₆, with the proviso none of the Zr-MOF(s) is/are Zr₆O₄(OH)₄(fumarate)₆ (Zr-fum) or Zr₆O₄(OH)₄(mesaconate)₆ (Zr-mes),
the method comprising:
forming a Zr-MOF reaction mixture comprising:
one or more zirconium compound(s),
one or more polycarboxylic acid(s), one or more polycarboxylate salt(s), or any combination thereof,
one or more basic solvent(s), one or more non-basic solvents(s), or any combination thereof, and
optionally, one or more acid modulator(s),
heating the reaction mixture, wherein one or more Zr-MOF(s) is/are formed, and
optionally, isolating and/or activating the Zr-MOF(s).

8. The method of making Zr-MOF(s) of claim 7, wherein the acid modulator(s) is/are chosen from carboxylic acids, amino acids, mineral acids, and any combination thereof

9. The method of making Zr-MOF(s) of claim 7, wherein the Zr-MOF reaction mixture is heated at a temperature of from about 30 degrees Celsius (° C.) to about 150° C.

10. A hydrogen sulfide-loaded Zr-based metal-organic framework (H₂S-loaded Zr-MOF) comprising a Zr-based metal organic framework (Zr-MOF) comprising the following formula:
Zr₆O₄(OH)₄(polycarboxylate)₆,
wherein the Zr-MOF comprises H₂S.

11. The H₂S-loaded Zr-MOF of claim 10, wherein the H₂S-loaded Zr-MOF comprises an H₂S loading of from about 0 millimol H₂S per gram of Zr-MOF (mmol H₂S/g Zr-MOF) to about 10 mmol H₂S/g Zr-MOF.

12. The H₂S-loaded Zr-MOF of claim 10, wherein the H₂S-loaded Zr-MOF is capable of releasing at least a portion of or all of the H₂S comprised in the H₂S-loaded Zr-MOF.

13. The H₂S-loaded Zr-MOF of claim 10, wherein the H₂S-loaded Zr-MOF exhibits a H₂S cycling capacity decrease after 10 cycles of from about 0% to about 8%.

14. A method of making one or more hydrogen sulfide-loaded metal organic framework(s) (H₂S-loaded Zr-MOF(s)), the method comprising:
forming an H₂S-loading reaction mixture comprising:
H₂S, and
one or more Zr-MOF(s) independently comprising the following formula:
Zr₆O₄(OH)₄(polycarboxylate)₆,
wherein one or more Zr-MOF(s) comprising H₂S is/are formed, and
optionally, isolating and/or activating the H₂S-loaded Zr-MOF(s).

15. The method of making H₂S-loaded Zr-MOF(s) claim 14, the method further comprising:
subjecting the H₂S-loaded Zr-MOF(s) to reduced pressure, heat, an aqueous environment, a solvent, or any combination thereof, thereby forming one or more desorbed Zr-MOF(s), and
optionally, isolating and/or activating the desorbed Zr-MOF(s),
wherein one or more desorbed Zr-MOF(s) are formed.

16. A method of H₂S delivery, the method comprising:
contacting an aqueous environment, a solvent, or any combination thereof, with one or more H₂S-loaded Zr-MOF(s), wherein at least a portion of or all of the H₂S comprised by the H₂S-loaded Zr-MOF(s) is released into the aqueous environment, the solvent, or the combination thereof, and wherein one or more desorbed Zr-MOF(s) is/are formed, and
optionally, isolating and/or activating the desorbed Zr-MOF(s).

17. The method of H₂S delivery of claim 16, wherein the H₂S-loaded Zr-MOF(s) deliver from about 0 micromol per liter (μM) H₂S to about 400 μM H₂S to the aqueous environment, the solvent, or the combination thereof.

18. The method of H₂S delivery of claim 16, wherein the aqueous environment is chosen from neutral deionized water, phosphate buffered saline (PBS), cell culture medium, fetal bovine serum (FBS), and any combination thereof.

19. The method of H₂S delivery of claim 17, wherein, the aqueous environment is comprised within an individual or a portion of an individual.

20. The method of H₂S delivery of claim 16, wherein the H₂S-loaded Zr-MOF(s) is/are taken up by a cell or a population of cells, and wherein H₂S is released within the cell or the population of cells.

21. The method of H₂S delivery of claim 20, wherein the cell or the population of cells exhibits greater than about 50% viability after the contacting of the cell or the population of cells with about 1 mg/ml or less of H₂S-loaded Zr-MOF(s).

22. The method of H₂S delivery of claim 19, wherein the individual is an individual suffering from or at risk of an ischemia-reperfusion injury, inflammation, a wound, or any combination thereof, and wherein delivery treats or prevents the ischemia-reperfusion injury, the inflammation, the wound, or the combination thereof, in the individual.
